(19) United States
(12) Patent Application Publication
(10) Pub. No.: US 2022/0155299 A1
(43) Pub. Date: May 19, 2022

(54) FLOW CYTOMETRIC METHOD FOR CHARACTERIZATION OF T-CELL IMPURITIES

(71) Applicant: Kite Pharma, Inc., Santa Monica, CA (US)

(72) Inventors: Qi Cai, North Hollywood, CA (US); Jonathan Kirzner, Los Angeles, CA (US); D.H. Tony Lee, West Hollywood, CA (US); Bharat Sowrirajan, Los Angeles, CA (US); Michelle Tseng, Marina del Rey, CA (US); Hemamali Warshakoon, Lawndale, CA (US)

(21) Appl. No.: 17/512,071

(22) Filed: Oct. 27, 2021

Related U.S. Application Data
(60) Provisional application No. 63/106,728, filed on Oct. 28, 2020.

(51) Int. Cl. G01N 33/569; G01N 21/64; G01N 33/68

(52) U.S. Cl. CPC G01N 33/56966; G01N 33/6854; G01N 21/6428

(57) ABSTRACT

Compositions and methods for fluorescence activated cell analysis of blood cell populations.

### CROSS-REFERENCE TO RELATED APPLICATIONS

This application claims the benefit under 35 U.S.C. § 119(e) of U.S. Provisional Application No. 63/106,728, filed on Oct. 28, 2020, which is hereby incorporated herein by reference in its entirety for all purposes.

### FIELD

Compositions and methods for fluorescence activated cell analysis of blood cell populations.

### BACKGROUND

T cell immunotherapy products that are prepared from blood samples contain non-T cell impurities such as B cells, NK cells, and monocytes. Given this cellular heterogeneity, multiple markers are needed to identify and characterize individual subsets. There is a need for methods to characterize and quantify the levels of those impurities at the different stages of the production process of T cell immunotherapy product and in the final product.

### SUMMARY

In one embodiment, the disclosure provides methods and compositions for flow cytometric quantitation of CD3− cellular impurities in lymphocyte-rich samples. In one embodiment, the disclosure provides fit-for-purpose 2-8 color T-cell impurity flow cytometry panels, which detect up to seven different blood cell surface markers and also include a viability dye. In one embodiment, the disclosure provides methods of using the panels for the detection and quantification of CD3− cells in samples obtained at different stages of manufacturing of a T cell product for immunotherapy. The panels and methods disclosed herein have several efficiency-promoting properties including, but not limited to, easy of use, elimination of the need of titrating different antibody lots, inclusion of lot matched isotype controls, lot to lot consistency, long term stability at room temperature, minimization of error prone pipetting steps and cocktailing, stream lined workflow, and improved data reliability. In one embodiment, the panels or methods may be used to characterize impurities in T cell populations for immunotherapy. The following are exemplary, non-limiting embodiments of this disclosure.

A method of simultaneous identifying two or more of lymphocytes, NK-T cells, NK cells, monocytes, early B cell progenitor cell, or combinations thereof in a cell population, comprising simultaneously detecting the presence or absence of two or more of lymphocytes, NK-T cells, NK cells, monocytes, and/or early B cell progenitor cells using two or more of the markers on the surface of these cells as described in Table 2, optionally with one or more of the fluorescently-labeled antibodies as described in Tables 3, 4, and 5, using fluorescence detection methods.

A method of assessing the non-CD3+ contaminants in a population of cells comprising primarily CD4+ and/or CD8+ T cells comprising contacting the population of cells with one or more antibodies against specific surface markers for lymphocytes, NK-T cells, NK cells, monocytes, and/or early B cell progenitor cell to create a mixture, wherein two or more of the specific cell surface markers are described in Table 2, optionally, wherein the one or more antibodies are selected from Tables 3, 4, and 5, and analyzing the mixture for the distribution of cells with specific cell surface markers by fluorescence detection methods.

A method of treating cancer in a subject by immunotherapy in need thereof, comprising administering to the subject a T cell preparation wherein one or more of the CD3-impurities (e.g., NK-T cells, NK cells, monocytes, early B cell progenitor cell, or combinations thereof) in the T cell preparation is characterized by the method of any one of embodiments 1 and 2.

In embodiments, the T cell preparation is autologous, optionally from a cancer patient or a healthy donor.

In embodiments, the T cell preparation is allogeneic, optionally from a cancer patient or a heathy donor.

In embodiments, the the T cells are engineered with a CAR or T cell receptor.

A method for determining whether a T cell product is suitable for immunotherapy, comprising characterizing one or more of the CD3− cell impurities (e.g., NK-T cells, NK cells, monocytes, early B cell progenitor cell, or combinations thereof) in the T cell product using one of the antibodies or cocktail of antibodies described in Tables 3, 4, and 5, and determining whether the T cell product is suitable based on the levels of CD3− cell impurities in the T cell product.

In embodiments, the acceptable levels are set by regulatory authorities (e.g., FDA, EMEA, etc).

An assay or a kit for identifying at least one of T lymphocytes, NK-T cells, NK cells, monocytes total lymphocytes, early B cell progenitor cell, or combinations thereof in a blood cell population using one or more of the antibodies or cocktails of antibodies described in Tables 3, 4, and 5.

In embodiments, the assay or kit is used to characterize the presence of CD3-cells in T cell products for immunotherapy.

In embodiments, the kit comprises (a) one of more antibodies to detect one or more cell surface markers for any one or more of these cells (see, e.g., Table 2) and (2) reagents to carry on the binding of the antibody with the cell surface markers, and, optionally, (3) instructions for using the reagents for the kit's purpose.

In embodiments, the antibodies (two or more) are all lyophilized together in the same container (e.g., a Lyovial).

In embodiments, the antibodies are selected from Table 3.

In embodiments, the antibodies are selected from Table 4.

In embodiments, the antibodies are selected from Table 5.

A composition comprising a panel of fluorescently-labeled antibodies for identifying the presence or absence of T cells, NK-T cells, NK cells, monocytes, early B cell progenitor cell, or combinations thereof cells in a cell population, comprising two or more antibodies against two or more of the cell surface markers identified in Table 2, optionally wherein one or more of the antibodies are described in Tables 3, 4, or 5.

In embodiments, the composition comprises all of the antibodies in Tables 3, 4, or 5, optionally, together with a cell viability marker.

In embodiments, the composition comprises antibodies against all of the cell surface markers in Table 2, optionally, together with a cell viability marker.

In embodiments, the composition comprises all of the antibodies described in Table 6 in the same amounts of Table 6 or in identical multiples of such amounts (e.g., all amounts equally doubled, tripled, quadrupled, etc.).

### BRIEF DESCRIPTION OF THE DRAWINGS

FIG. 1A and FIG. 1B Antibody titrations and individual scatter-grams for Anti-CD3 APC (A) and Anti-CD14 PerCP-Cy5.5 (B).

FIGS. 2A and 2B show FACS staining comparison between assays with liquid using a healthy donor (FIG. 2A Flow cytometry antibody staining panel-CD10-FITC/CD56-PE/CD14-PerCP-Cy5.5/CD19-PECy7/CD3-APC/CD34-BV421/CD45-V500) cocktail and lyophilized (FIG. 2B Flow cytometry antibody staining panel-CD10-FITC/CD56-PE/CD14-PerCP-Cy5.5/CD19-PECy7/CD3-APC/CD34-BV421/CD45-V500) reagent).

FIG. 3A shows an example of FACS staining of a clinical sample using lyophilized reagent and FIG. 3B shows the 10 day stability of the lyophized reagents using patients and healthy donor samples. FIG. 3B illustrates the data from Table 16.

FIG. 4 shows that inter-assay precision was optimal.

FIG. 5 Method specificity: plots for CD34, CD19, and CD56 antibody detection evaluations.

FIG. 6 Method robustness: antibody staining incubation time.

### DETAILED DESCRIPTION

### Definitions

Except as otherwise expressly provided herein, each of the following terms shall have the meaning set forth below. Additional definitions are set forth throughout the application. Unless defined otherwise, all technical and scientific terms used herein have the meaning as commonly understood by one of ordinary skill in the art. For example, the manual Current Protocols In Immunology, edited by John E. Coligan, Ada M. Kruisbeek, David H. Margulies, Ethan M. Shevach, Warren Strober, (Series Editior: Richard Coico), ISBN 0471522767; Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2006, Oxford University Press, provide one of skill with a general dictionary of many of the terms used in this application.

Units, prefixes, and symbols are denoted in their Systeme International de Unites (SI) accepted form. Numeric ranges are inclusive of the numbers defining the range. The disclosure provided herein are not limitations of the various aspects of the application, which may be by reference to the specification as a whole. Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

The articles “a” or “an” refer to “one or more” of any recited or enumerated component.

The terms “about” or “comprising essentially of” refer to a value or composition that is within an acceptable error range for certain value or composition as determined by one of ordinary skill in the art, which will depend in part on how the value or composition is measured or determined, i.e., the limitations of the measurement system. For example, “about” or “comprising essentially of” may mean within 1 or more than 1 standard deviation per the practice in the art. Alternatively, “about” or “comprising essentially of” may mean a range of up to 10% (i.e., ±10%). For example, about 3 mg may include any number between 2.7 mg and 3.3 mg (for 10%). With respect to biological systems or processes, the terms may mean up to an order of magnitude or up to 5-fold of a value. When certain values or compositions are provided in the application and claims, unless otherwise stated, the meaning of “about” or “comprising essentially of” include an acceptable error range for that value or composition. Any concentration range, percentage range, ratio range, or integer range includes the value of any integer within the recited range and, when appropriate, fractions thereof (such as one-tenth and one-hundredth of an integer), unless otherwise indicated. As an example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

Unless specifically stated or obvious from context, as used herein, the term “or” is understood to be inclusive and covers both “or” and “and”. The term “and/or” refer to each of the two specified features or components with or without the other. Thus, the term “and/or” as used in a phrase such as “A and/or B” herein is intended to include “A and B,” “A or B,” “A” (alone), and “B” (alone). Similarly, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” are used merely by way of example, without limitation intended, and not be construed as referring only those items explicitly enumerated in the specification.

The terms “or more”, “at least”, “more than”, and the like, e.g., “at least one” include but not be limited to at least 1, 2, 3, 4, 5, 6, 7, 8, 9, 10, 11, 12, 13, 14, 15, 16, 17, 18, 19 20,21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. The term “no more than” includes each value less than the stated value. For example, “no more than xyx” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 xyz. Also included is any lesser number or fraction in between.

The terms “plurality”, “at least two”, “two or more”, “at least second”, and the like include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” is understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

The terms “administration,” “Administering” or the like refer to physical introduction of an agent to a subject, using any of the various methods and delivery systems known to those skilled in the art. Exemplary routes of administration for the immune cells prepared by the methods disclosed herein include intravenous (i.v. or IV), intramuscular, subcutaneous, intraperitoneal, spinal or other parenteral routes of administration, for example by injection or infusion. Parenteral route of administration refer to modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the immune cells (e.g., T cells) prepared by the present methods are administered via injection or infusion. Non-parenteral routes include a topical, epidermal or mucosal route of administration, for example, intranasally, vaginally, rectally, sublingually or topically. Administering may also be once, twice, or a plurality of times over one or more extended periods. Where one or more therapeutic agents (e.g., cells) are administered, the administration may be done concomitantly or sequentially. Sequential administration comprises administration of one agent only after administration of the other agent or agents has been completed.

The term “antibody” (Ab) includes, without limitation, an immunoglobulin which binds specifically to an antigen. In general, an antibody may comprise at least two heavy (H) chains and two light (L) chains interconnected by disulfide bonds. Each H chain comprises a heavy chain variable region (abbreviated herein as VH) and a heavy chain constant region. The heavy chain constant region may comprise three or four constant domains, CH1, CH2 CH3, and/or CH4. Each light chain comprises a light chain variable region (abbreviated herein as VL) and a light chain constant region. The light chain constant region may comprise one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, a monovalent and a divalent fragment or portion, and a single chain Ab.

An “antigen binding molecule,” “antibody fragment” or the like refer to any portion of an antibody less than the whole. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. In one aspect, the CD19 CAR construct comprises an anti-CD 19 single-chain FV. A “Single-chain Fv” or “scFv” antibody binding fragment comprises the variably heavy (VH) and variable light (V_{L}) domains of an antibody, where these domains are present in a single polypeptide chain. Generally, the Fv polypeptide further comprises a polypeptide linker between the V_{H} and V_{L} domains, which enables the scFv to form the desired structure for antigen binding. All antibody-related terms used herein take the customary meaning in the art and are well understood by one of ordinary skill in the art.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e., expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy method described herein involves a collection of lymphocytes from an individual (such as a donor or a patient), which are then engineered to express a CAR construct and then administered back to the same individual.

The term “allogeneic” refers to any material derived from one individual which is then introduced to another individual of the same species, e.g., allogeneic T cell transplantation.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor at various stages. In one embodiment, the cancer or tumor is stage 0, such that, e.g., the cancer or tumor is very early in development and has not metastasized. In another embodiment, the cancer or tumor is stage I, such that, e.g., the cancer or tumor is relatively small in size, has not spread into nearby tissue, and has not metastasized. In other embodiment, the cancer or tumor is stage II or stage III, such that, e.g., the cancer or tumor is larger than in stage 0 or stage I, and it has grown into neighboring tissues but it has not metastasized, except potentially to the lymph nodes. In additional embodiment, the cancer or tumor is stage IV, such that, e.g., the cancer or tumor has metastasized. Stage IV may also be referred to as advanced or metastatic cancer.

In certain embodiments, the cancer may be selected from a tumor derived from acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

In one embodiment, the method may be used to treat a tumor, wherein the tumor is a lymphoma or a leukemia. Lymphoma and leukemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukemias may affect one or more of these cell types in a patient.

In general, lymphomas may be divided into at least two sub-groups: Hodgkin lymphoma and non-Hodgkin lymphoma. Non-Hodgkin Lymphoma (NHL) is a heterogeneous group of cancers originating in B lymphocytes, T lymphocytes or natural killer cells. In the United States, B cell lymphomas represent 80-85% of cases reported. In 2013 approximately 69,740 new cases of NHL and over 19,000 deaths related to the disease were estimated to occur. Non-Hodgkin lymphoma is the most prevalent hematological malignancy and is the seventh leading site of new cancers among men and women and account for 4% of all new cancer cases and 3% of deaths related to cancer.

In some embodiments, the method may be used to treat a lymphoma or a leukemia, wherein the lymphoma or leukemia is a B cell malignancy. Examples of B cell malignancies include, but are not limited to, Non-Hodgkin's Lymphomas (NHL), Small lymphocytic lymphoma (SLL/CLL), Mantle cell lymphoma (MCL), FL, Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, and Lymphoblastic lymphoma. In some aspects, the lymphoma or leukemia is selected from B-cell chronic lymphocytic leukemia/small cell lymphoma, B-cell prolymphocytic leukemia, lymphoplasmacytic lymphoma (e.g., Waldenström macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma, Lymphomatoid papulosis, Peripheral T-cell lymphoma, Angioimmunoblastic T cell lymphoma, Anaplastic large cell lymphoma, B-lymphoblastic leukemia/lymphoma, B-lymphoblastic leukemia/lymphoma with recurrent genetic abnormalities, T-lymphoblastic leukemia/lymphoma, and Hodgkin lymphoma. In some aspect, the cancer is refractory to one or more prior treatments, and/or the cancer has relapsed after one or more prior treatments.

In one embodiment, the cancer is selected from follicular lymphoma, transformed follicular lymphoma, diffuse large B cell lymphoma, and primary mediastinal (thymic) large B-cell lymphoma. In another embodiment, the cancer is diffuse large B cell lymphoma. In some embodiment, the cancer is refractory to or the cancer has relapsed following one or more of chemotherapy, radiotherapy, immunotherapy (including a T cell therapy and/or treatment with an antibody or antibody-drug conjugate), an autologous stem cell transplant, or any combination thereof. In one embodiment, the cancer is refractory diffuse large B cell lymphoma or mantle cell lymphoma.

An “anti-tumor effect” as used herein, refers to a biological effect that may present, and not being limited to, as a decrease in tumor volume, an inhibition of tumor growth, a decrease in the number of tumor cells, a decrease in tumor cell proliferation, a decrease in the number/extent of metastases, an increase in overall or progression-free survival, an increase in life expectancy, and/or amelioration of various physiological symptoms associated with the tumor. An anti-tumor effect may also refer to the prevention of the occurrence of a tumor, e.g., a vaccine.

A “therapeutically effective amount,” “therapeutically effective dosage,” or the like refers to an amount of the cells (such as immune cells or engineered T cells) that are produced by the present methods (resulting in a T cell product) and that, when used alone or in combination with another therapeutic agent, protects or treats a subject against the onset of a disease or promotes disease regression as evidenced by a decrease in severity of disease symptoms, an increase in frequency and duration of disease symptom-free periods, and/or prevention of impairment or disability due to disease affliction. The ability to promote disease regression may be evaluated using a variety of methods known to the skilled practitioner, such as in subjects during clinical trials, in animal model systems predictive of efficacy in humans, or by assaying the activity of the agent in in vitro assays. In some embodiments, the donor T cells for use in the T cell therapy are obtained from the patient (e.g., for an autologous T cell therapy). In other embodiments, the donor T cells for use in the T cell therapy are obtained from a subject that is not the patient. The T cells may be administered at a therapeutically effective amount. For example, a therapeutically effective amount of the T cells, e.g., engineered CAR+ T cells or engineered TCR+ T cells, may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells. In some embodiments, the therapeutically effective amount of the CAR-positive viable T cells is between about 0.4×10⁸ and about 2×10⁸ CAR-positive viable T cells. In some embodiments, the therapeutically effective amount of the CAR-positive viable T cells is about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10⁸, or about 2.0×10⁸ CAR-positive viable T cells.

The term “lymphocyte” as used herein may include natural killer (NK) cells, T cells, NK-T cells, or B cells. NK cells are a type of cytotoxic (cell toxic) lymphocyte that represent a major component of the inherent immune system. NK cells reject tumors and cells infected by viruses, through the process of apoptosis or programmed cell death. They were termed “natural killers” because they do not require activation to kill cells. T-cells play a major role in cell-mediated immunity (no antibody involvement). The T-cell receptors (TCR) differentiate themselves from other lymphocyte types. The thymus, a specialized organ of the immune system, is primarily responsible for the T cell's maturation.

There are several types of “immune cells,” including, without limitation, macrophages (e.g., tumor associated macrophages) neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells. The term also includes precursors of these immune cells. Hematopoietic stem and/or progenitor cells may be derived from bone marrow, umbilical cord blood, adult peripheral blood after cytokine mobilization, and the like, by methods known in the art. Some precursor cells are those that may differentiate into the lymphoid lineage, for example, hematopoietic stem cells or progenitor cells of the lymphoid lineage. Additional examples of immune cells that may be used for immune therapy are described in US Publication No. 20180273601, incorporated herein by reference in its entirety.

There are also several types of T-cells, namely: Helper T-cells (e.g., CD4+ cells, effector T_{EFF} cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and are CCR7T and CD45RO′ and they secrete IL-2, but not IFNγ or IL-4, and (iii) effector memory T_{EM} cells, however, do not express L-selectin or CCR7 but do express CD45RO and produce effector cytokines like IFNγ and IL-4), Regulatory T-cells (Tregs, suppressor T cells, or CD4+CD25+ regulatory T cells), Natural Killer T-cells (NKT), and Gamma Delta T-cells. T cells found within tumors are referred to as “tumor infiltrating lymphocytes” (TIL). B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

A “naïve” T cell refers to a mature T cell that remains immunologically undifferentiated. Following positive and negative selection in the thymus, T cells emerge as either CD4⁺ or CD8⁺ naïve T cells. In their naïve state, T cells express L-selectin (CD62L⁻), IL-7 receptor-α (IL-7R-α), and CD132, but they do not express CD25, CD44, CD69, or CD45RO. As used herein, “immature” may also refer to a T cell which exhibits a phenotype characteristic of either a naïve T cell or an immature T cell, such as a T_{SCM} cell or a T_{CM} cell. For example, an immature T cell may express one or more of L-selectin (CD62L⁺), IL-7Rα, CD132, CCR7, CD45RA, CD45RO, CD27, CD28, CD95, IL-2Rβ, CXCR3, and LFA-1. Naïve or immature T cells may be contrasted with terminal differentiated effector T cells, such as T_{EM} cells and T_{EFF} cells.

The terms cell “proliferation,” “proliferating” or the like refer to the ability of cells to grow in numbers through cell division. Proliferation may be measured by staining cells with carboxyfluorescein succinimidyl ester (CFSE). Cell proliferation may occur in vitro, e.g., during T cell culture, or in vivo, e.g., following administration of a immune cell therapy (e.g., T cell therapy). The cell proliferation may be measured or determined by the methods described herein or known in the field. For example, cell proliferation may be measured or determined by viable cell density (VCD) or total viable cell (TVC). VCD or TVC may be theoretical (an aliquot or sample is removed from a culture at certain timepoint to determine the cell number, then the cell number multiples with the culture volume at the beginning of the study) or actual (an aliquot or sample is removed from a culture at certain timepoint to determine the cell number, then the cell number multiples with the actual culture volume at the certain timepoint). The term “T cell activity” refers to any activity common to healthy T cells. In one embodiment, the T cell activity comprises cytokine production (such as INFγ, IL-2, and/or TNFα). In other embodiment, the T cell activity comprises production of one or more cytokine selected from interferon gamma (IFNγ or IFN-γ), tissue necrosis factor alpha (TNFα or IFNα), and both. The terms “cytolytic activity,” “cytotoxicity” or the like refer to the ability of a T cell to destroy a target cell. In one embodiment, the target cell is a cancer cell, e.g., a tumor cell. In other embodiments, the T cell expresses a chimeric antigen receptor (CAR) or a T cell receptor (TCR), and the target cell expresses a target antigen.

The term “genetically engineered,” “gene editing,” or “engineered” refers to a method of modifying the genome of a cell, including, but not being limited to, deleting a coding or non-coding region or a portion thereof or inserting a coding region or a portion thereof. In one embodiment, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Chimeric antigen receptors” (CARs or CAR-Ts) and the T cell receptors (TCRs) of the application are genetically engineered receptors. These engineered receptors may be readily inserted into and expressed by immune cells, including T cells, in accordance with techniques known in the art. With a CAR, a single receptor may be programmed to both recognize a specific antigen and, when bound to that antigen, activate the immune cell to attack and destroy the cell bearing or expressing that antigen. When these antigens exist on tumor cells, an immune cell that expresses the CAR may target and kill the tumor cell. In one embodiment, the cell that are prepared according to the present application is a cell having a chimeric antigen receptor (CAR), or a T cell receptor, comprising an antigen binding molecule, one or more costimulatory domains, and one or more activating domains. The costimulatory domain may comprise an extracellular domain, a transmembrane domain, and an intracellular domain. In one embodiment, the extracellular domain comprises a hinge or a truncated hinge domain.

The “antigen binding molecule” may comprise a binding molecule to a tumor antigen. The binding molecule may be an antibody or an antigen binding molecule thereof. For example, the antigen binding molecule may be selected from scFv, Fab, Fab′, Fv, F(ab′)2, and dAb, and any fragments or combinations thereof. The chimeric antigen receptor may further comprise a hinge region. The hinge region may be derived from the hinge region of IgG1, IgG2, IgG3, IgG4, IgA, IgD, IgE, IgM, CD28, or CD8 alpha. In one embodiment, the hinge region is derived from the hinge region of IgG4. The chimeric antigen receptor may also comprise a transmembrane domain. The transmembrane domain may be a transmembrane domain of any transmembrane molecule that is a co-receptor on immune cells or a transmembrane domain of a member of the immunoglobulin superfamily. In certain embodiment, the transmembrane domain is derived from a transmembrane domain of CD28, CD28T, CD8 alpha, CD4, or CD19. In another embodiment, the transmembrane domain comprises a domain derived from a CD28 transmembrane domain. In another embodiment, the transmembrane domain comprises a domain derived from a CD28T transmembrane domain.

The “antigen” may be the tumor antigen selected from 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), L1CAM (L1 cell adhesion molecule), MAGE (melanoma antigen), MAGE-A1 (Melanoma-associated antigen 1), MAGE-A3, MAGE-A6, mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), WT1 (Wilms' tumor gene), and any combination thereof. In one embodiment, the tumor antigen is CD19.

In one embodiment, the T cell products of the disclosure are used in “CD19-directed genetically modified autologous T cell immunotherapy,” which refers to a suspension of chimeric antigen receptor (CAR)-positive immune cells. An example of such immunotherapy is Clear CAR-T therapy, which uses CAR-T cells that are free of circulating tumor cells and enriched in CD4+/CD8+ T cells. Another example is axicabtagene ciloleucel (also known as Axi-cel™, YESCARTA©). See Kochenderfer, et al., (J Immunother 2009; 32:689 702). In one embodiment, the T cell product is brexucabtagene autoleucel (formerly KTE-X19; Tecartus) Other non-limiting examples include JCAR017, JCAR015, JCAR014, Kymriah (tisagenlecleucel), Uppsala U. anti-CD19 CAR (NCT02132624), and UCART19 (Celectis). See Sadelain et al. Nature Rev. Cancer Vol. 3 (2003), Ruella et al., Curr Hematol Malig Rep., Springer, N.Y. (2016) and Sadelain et al. Cancer Discovery (April 2013) To prepare CD19-directed genetically modified autologous T cell immunotherapy, a patient's own T cells may be harvested and genetically modified ex vivo by retroviral transduction to express a chimeric antigen receptor (CAR) comprising a murine anti-CD19 single chain variable fragment (scFv) linked to CD28 and CD3-zeta co-stimulatory domains. In some embodiments, the CAR comprises a murine anti-CD19 single chain variable fragment (scFv) linked to 4-1BB and CD3-zeta co-stimulatory domain. The anti-CD19 CAR T cells may be expanded and infused back into the patient, where they may recognize and eliminate CD19-expressing target cells.

In some embodiments, the T cells are engineered with a T cell receptor (TCR), which may comprise a binding molecule to a tumor antigen. In some aspects, the tumor antigen is selected from the group consisting of 707-AP, AFP, ART-4, BAGE, BCMA, Bcr-abl, CAIX, CD19, CD20, CD22, CD30, CD33, CD44v7/8, CAMEL, CAP-1, CASP-8, CDCl27m, CDK4/m, CEA, CT, Cyp-B, DAM, EGFR, EGFRvIII, EGP-2, EGP-40, Erbb2, 3, 4, ELF2M, ETV6-AML1, FBP, fAchR, G250, GAGE, GD2, GD3, GnT-V, Gp100, HAGE, HER-2/neu, HLA-A, HPV, HSP70-2M, HST-2, hTERT or hTRT, iCE, IL-13R-a2, KIAA0205, KDR, κ-light chain, LAGE, LDLR/FUT, LeY, LlCAM, MAGE, MAGE-A1, mesothelin, Murine CMV infected cells, MART-1/Melan-A, MC1R, Myosin/m, MUC1, MUM-1, -2, -3, NA88-A, NKG2D ligands, NY-BR-1, NY-ESO-1, oncofetal antigen, P15, p190 minor bcr-abl, Pml/RARa, PRAME, PSA, PSCA, PSMA, RAGE, RU1 or RU2, SAGE, SART-1 or SART-3, SSX1, -2, -3, 4, TAA, TAG-72, TEL/AML1, TPI/m, TRP-1, TRP-2, TRP-2/INT2, VEGF-R2, WTi, and any combination thereof. In one aspect, the TCR comprises a binding molecule to a viral oncogene. In one embodiment, the viral oncogene is selected from human papilloma virus (HPV), Epstein-Barr virus (EBV), and human T-lymphotropic virus (HTLV). In other embodiments, the TCR comprises a binding molecule to a testicular, placental, or fetal tumor antigen. In one embodiment, the testicular, placental, or fetal tumor antigen is selected from the group consisting of NY-ESO-1, synovial sarcoma X breakpoint 2 (SSX2), melanoma antigen (MAGE), and any combination thereof. In another embodiment, the TCR comprises a binding molecule to a lineage specific antigen. In additional embodiment, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and any combination thereof. In certain embodiment, the T cell therapy comprises administering to the patient engineered CAR T cells expressing a chimeric antigen receptor that binds to CD19 and further comprises a CD28 costimulatory domain and a CD3-zeta signaling region. In additional embodiment, the T cell therapy comprises administering to a patient KTE-C19 or KTE-X19. In one aspect, the antigenic moieties also include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL. gM, UL20, UL32, US43, UL45, UL49A), a cytomegalovirus (CMV) viral antigen (e.g., glycoproteins gB, gC, gC, gE, gG, gH, gI, gJ, gK, gL. gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such aspects, the cell surface receptor may be any TCR, or any CAR which recognizes any of the aforementioned viral antigens on a target virally infected cell. In other aspects, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

The “costimulatory domain” may be a signaling region derived from, e.g., CD28, CTLA4, CD16, OX-40, 4-1BB/CD137, CD2, CD7, CD27, CD30, CD40, programmed death-1 (PD-1), programmed death ligand-1 (PD-L1), inducible T cell costimulator (ICOS), ICOS-L, lymphocyte function-associated antigen-1 (LFA-1 (CD11a/CD18), CD3 gamma, CD3 delta, CD3 epsilon, CD247, CD276 (B7-H3), LIGHT (tumor necrosis factor superfamily member 14; TNFSFi4), NKG2C, Ig alpha (CD79a), DAP-10, Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGBI, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The “activating domain” may be derived from, e.g., CD3, such as CD3 zeta, epsilon, delta, gamma, or the like. In one embodiment, the CAR is designed to have two, three, four, or more costimulatory domains.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The terms “immunotherapy” “immune therapy” or the like refer to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell and NK cell therapies. T cell therapy may include adoptive T cell therapy, tumor-infiltrating lymphocyte (TIL) immunotherapy, autologous cell therapy, engineered autologous cell therapy and allogeneic T cell transplantation. One of skill in the art would recognize that the methods of preparing immune cells disclosed herein would enhance the effectiveness of any cancer or transplanted T cell therapy. Examples of T cell therapies are described in U.S. Patent Publication Nos. 2014/0154228 and 2002/0006409; U.S. Pat. Nos. 7,741,465; 6,319,494; and 5,728,388; and PCT Publication No. WO 2008/081035, which are incorporated by reference in their entirety.

The one or more immune cells described herein may be obtained from any source, including, for example, a human donor. The donor may be a subject in need of an anti-cancer treatment, e.g., treatment with one immune cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). immune cells may be differentiated in vitro from a hematopoietic stem cell population, or immune cells may be obtained from a donor. The population of immune cells may be obtained from the donor by any suitable method used in the art. For example, the population of lymphocytes may be obtained by any suitable extracorporeal method, venipuncture, or other blood collection method by which a sample of blood with or without lymphocytes is obtained. The population of lymphocytes is obtained by apheresis. The one or more immune cells may be collected from any tissue that comprises one or more immune cells, including, but not limited to, a tumor. A tumor or a portion thereof is collected from a subject, and one or more immune cells are isolated from the tumor tissue. Any T cell may be used in the methods disclosed herein, including any immune cells suitable for a T cell therapy. For example, the one or more cells useful for the application may be selected from the group consisting of tumor infiltrating lymphocytes (TIL), cytotoxic T cells, CAR T cells, engineered TCR T cells, natural killer T cells, Dendritic cells, and peripheral blood lymphocytes. T cells may be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. T cells may also be obtained from an artificial thymic organoid (ATO) cell culture system, which replicates the human thymic environment to support efficient ex vivo differentiation of T-cells from primary and reprogrammed pluripotent stem cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entirety. In one embodiment, T cells are tumor infiltrating leukocytes. In certain embodiment, the one or more T cells express CD8, e.g., are CD8+ T cells. In other embodiment, the one or more T cells express CD4, e.g., are CD4+ T cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entiretyIn some aspect, the cells of the present application may be obtained through T cells obtained from a subject. In one aspect, the T cells may be obtained from, e.g., peripheral blood mononuclear cells (PBMC), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. In some aspect, the cells collected by apheresis are washed to remove the plasma fraction and placed in an appropriate buffer or media for subsequent processing. In some aspect, the cells are washed with any solution (e.g., a solution with neutralized PH value or PBS) or culture medium. As will be appreciated, a washing step may be used, such as by using a semiautomated flow through centrifuge, e.g., the Cobe™ 2991 cell processor, the Baxter CytoMate™, or the like. In some aspect, the washed cells are resuspended in one or more biocompatible buffers, or other saline solution with or without buffer. In some aspect, the undesired components of the apheresis sample are removed. Additional methods of isolating T cells for a T cell therapy are disclosed in U.S. Patent Pub. No. 2013/0287748, which are hereby incorporated by references in their entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+, CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In one embodiment, CD3+ T cells are isolated from PBMCs using Dynabeads coated with anti-CD3 antibody. CD8+ and CD4+ T cells are further separately isolated by positive selection using CD8 microbeads (e.g., Miltenyi Biotec) and/or CD4 microbeads (e.g., Miltenyi Biotec).

PBMCs may be used directly for genetic modification with the immune cells (such as CARs). After isolating the PBMCs, T lymphocytes are further isolated, and both cytotoxic and helper T lymphocytes are sorted into naive, memory, and effector T cell subpopulations either before or after genetic modification and/or expansion. In one embodiment, CD8+ cells may be further sorted into naive, central memory, and effector cells by identifying cell surface antigens that are associated with each of these types of CD8+ cells. In other embodiment, the expression of phenotypic markers of central memory T cells includes CCR7, CD3, CD28, CD45RO, CD62L, and CD127 and are negative for granzyme B. In some embodiment, central memory T cells are CD8+, CD45RO+, and CD62L+ T cells. In certain embodiment, effector T cells are negative for CCR7, CD28, CD62L, and CD127 and positive for granzyme B and perforin. In additional embodiment, CD4+ T cells may be further sorted into subpopulations. For example, CD4+T helper cells may be sorted into naive, central memory, and effector cells by identifying cell populations that have cell surface antigens.

The methods described herein further comprise enriching or preparing a population of immune cells obtained from a donor, between harvesting from the donor and exposing one or more cells obtained from a donor subject. Enrichment of a population of immune cells, e.g., the one or more T cells, may be accomplished by any suitable separation method including, but not limited to, the use of a separation medium (e.g., FICOLL-PAQUE™, ROSETTESEP™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation.

In one embodiment, the T cell preparations described herewith may be used for engineered Autologous Cell Therapy. The term “engineered Autologous Cell Therapy,” which may be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR) or T cell receptor (TCR). CAR positive (+) T cells are engineered to express an extracellular single chain variable fragment (scFv) with specificity for certain tumor antigen linked to an intracellular signaling part comprising a costimulatory domain and an activating domain.

In some embodiments, the donor T cells for use in the T cell therapy are obtained from the patient (e.g., for an autologous T cell therapy). In other embodiments, the donor T cells for use in the T cell therapy are obtained from a subject that is not the patient. The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells. In some embodiments, the therapeutically effective amount of the CAR-positive viable T cells is between about 0.4×10⁸ and about 2×10⁸ CAR-positive viable T cells. In some embodiments, the therapeutically effective amount of the CAR-positive viable T cells is about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10⁸, or about 2.0×10⁸ CAR-positive viable T cells.

A “patient” as used herein includes any human who is afflicted with a disease or disorder, including cancer (e.g., a lymphoma or a leukemia). The terms “subject” and “patient” are used interchangeably herein. The term “donor subject” refers to herein a subject whose cells are being obtained for further in vitro engineering. The donor subject may be a cancer patient that is to be treated with a population of cells generated by the methods described herein (i.e., an autologous donor), or may be an individual who donates a lymphocyte sample that, upon generation of the population of cells generated by the methods described herein, will be used to treat a different individual or cancer patient (i.e., an allogeneic donor). Those subjects who receive the cells that were prepared by the present methods may be referred to as “recipient subject.”

The patients may be preconditioned or lymphodepleted prior to administration of the T cell therapy. The patient may be preconditioned according to any methods known in the art, including, but not limited to, treatment with one or more chemotherapy drug and/or radiotherapy. In some aspects, the preconditioning may include any treatment that reduces the number of endogenous lymphocytes, removes a cytokine sink, increases a serum level of one or more homeostatic cytokines or pro-inflammatory factors, enhances an effector function of T cells administered after the conditioning, enhances antigen presenting cell activation and/or availability, or any combination thereof prior to a T cell therapy. The preconditioning may comprise increasing a serum level of one or more cytokines in the subject. The methods further comprise administering a chemotherapeutic. The chemotherapeutic may be a lymphodepleting (preconditioning) chemotherapeutic. Beneficial preconditioning treatment regimens, along with correlative beneficial biomarkers are described in U.S. Pat. No. 9,855,298, which is hereby incorporated by reference in its entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient. In one aspect, the conditioning regimen comprises cyclophosphamide 500 mg/m²+fludarabine 30 mg/m² for 3 days. They may be administered at days −4, −3, and −2 or at days −5, −4, and −3 (day 0 being the day of administration of the cells). In one embodiment, the conditioning regimen comprises cyclophosphamide 200 mg/m², 250 mg/m², 300 mg/m², 400 v, 500 mg/m² daily for 2, 3, or 4 days and fludarabine 20 mg/m², 25 mg/m², or 30 mg/m² for 2, 3, or 4 days. In one embodiment, and after leukapheresis, conditioning chemotherapy (fludarabine 30 mg/m²/day and cyclophosphamide 500 mg/m²/day) is administered on days −5, −4, and −3 prior to an intravenous infusion of a suspension of CD19 CAR-T cells. In some embodiments, the intravenous infusion time is between 15 and 120 minutes. In one embodiment, the intravenous infusion time is between 1 and 240 minutes. In some embodiments, the intravenous infusion time is up to 30 minutes. In some embodiments, the intravenous infusion time is up to 5, 10, 15, 20, 25, 30, 35, 40, 45, 50, 55, 60, 65, 70, 75, 80, 85, 90, 95, or up to 100 minutes. In some embodiments, the infusion volume is between 50 and 100 mL. In some embodiments, the infusion volume is between 20 and 100 ml. In some embodiments, the infusion volume is about 30, 35, 40, 45, 50, 55, 60, or about 65 ml. In some embodiments, the infusion volume is about 68 mL. In some embodiments, the suspension has been frozen and is used within 6, 5, 4, 3, 2, 1 hour of thawing. In some embodiments, the suspension has not been frozen. In some embodiments, the immunotherapy is infused from an infusion bag. In some embodiments, the infusion bag is agitated during the infusion. In some embodiments, the immunotherapy is administered within 3 hours after thawing. In some embodiments, the suspension further comprises albumin. In some embodiments, albumin is present in an amount of about 2-3% (v/v). In some embodiments, albumin is present in an amount of about 2.5% (v/v). In some embodiments, the albumin is present in an amount of about 1%, 2%, 3%, 4%, or 5% (v/v). In some embodiments, albumin is human albumin. In some embodiments, the suspension further comprises DMSO. In some embodiments, DMSO is present in an amount of about 4-6% (v/v). In some embodiments, DMSO is present in an amount of about 5% (v/v). In some embodiments, the DMSO is present in an amount of 1%, 2%, 3%, 4%, 5%, 6%, 7%, 8%, 9%, or 10% (v/v).

As used herein, the term “in vitro cell” refers to any cell which is cultured ex vivo. In one embodiment, an in vitro cell includes a T cell.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. The term “modulating” T cell maturation, as used herein, refers to the use of any intervention described herein to control the maturation and/or differentiation of one or more cells such as T cells. For example, modulating refers to inactivating, delaying or inhibiting T cell maturation. In another example, modulating refers to accelerating or promoting T cell maturation. The term “delaying or inhibiting T cell maturation” refers to maintaining one or more T cells in an immature or undifferentiated state. For example, “delaying or inhibiting T cell maturation” may refer to maintaining T cells in a naïve or T_{CM} state, as opposed to progressing to a T_{EM} or T_{EFF} state. In addition, “delaying or inhibiting T cell maturation” may refer to increasing or enriching the overall percentage of immature or undifferentiated T cells (e.g., naïve T cells and/or T_{CM} cells) within a mixed population of T cells. The state of a T cell (e.g., as mature or immature) may be determined, e.g., by screening for the expression of various genes and the presence of various proteins expressed on the surface of the T cells. For example, the presence of one or more marker selected from the group consisting of L-selectin (CD62L+), IL-7R-α, CD132, CR7, CD45RA, CD45RO, CD27, CD28, CD95, IL-2Rβ, CXCR3, LFA-1, and any combination thereof may be indicative of less mature, undifferentiated T cells.

“Treatment” or “treating” of a subject/patient refers to any type of intervention or process performed on, or the administration of one or more T cells prepared by the present application to, the subject/patient with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

Additional terms referred to in the EXAMPLES section of this disclosure are defined below in Table 1.

TABLE 1
Terms used in the EXAMPLES

| Term | Definition |
|---|---|
| ALL | Acute Lymphocytic Leukemia |
| NHL | Non Hodgkin's Lymphoma |
| Allophycocyanin | An intensely bright phycobiliprotein isolated from red |
| (APC) | algae. It has excitation/emission spectrum peak |
| | wavelengths of 594-633/660 nm. |
| Brilliant Violet | A polymer-based dye with excitation/emission |
| 421 (BV421) | spectrum peak wavelengths of 407/421 nm. |
| CV | Coefficient of variation |
| STDEV | Standard Deviation |
| FC | Flow cytometry |
| Fluorescein | A bright green fluorophore with excitation/emission |
| isothiocyanate | spectrum peak wavelengths of 494/520 nm. |
| (FITC) | |
| LLOQ | Lower limit of quantitation |
| MFI | Median fluorescent intensity |
| Near-IR Dead | Viability dye used to determine the viability of cells. |
| Cell Stain Kit | It has excitation/ emission spectrum peak wavelengths |
| | of 633/750 nm. |
| NK/NKT cells | Natural killer cells or natural killer T cells |
| PE-Cy7 | A tandem conjugate that combines PE and a cyanine |
| | dye Cy7. It has excitation/emission spectrum peak |
| | wavelengths of 496/785 nm. |
| PerCP-Cy5.5 | A tandem conjugate that combines a protein complex |
| | called PerCP and a cyanine dye Cy5.5. It has |
| | excitation/emission spectrum peak wavelengths of |
| | 482/676 nm. |
| PBMC | Peripheral blood mono-nuclear cells, any peripheral |
| | blood cell having a round nucleus |
| Phycoerythrin | An intensely bright phycobiliprotein isolated from red |
| (PE) | algae. It has excitation/emission spectrum peak |
| | wavelengths of 488-561/578 nm. |
| TVC | Total Viable Cells. TVC represents the number of |
| | viable cells in a given sample. TVC is calculated with |
| | results from NucleoCounter. |
| V500 | An organic dye with excitation/emission spectrum |
| | peak wavelengths of 415/500 nm. |

Various aspects of the application are described in further detail in the following subsections.

In one embodiment, the disclosure provides methods and compositions for flow cytometric quantitation of CD3− cellular impurities in lymphocyte-rich samples. In one embodiment, the disclosure provides fit-for-purpose 2-8 color T-cell impurity flow cytometry panels of antibodies. In one embodiment, one or more of the antibodies described in those panels is combined into a cocktail of antibodies for identifying CD3− cell impurities in a T cell sample. In one embodiment, the antibody cocktail is lyophilized. In one embodiment, the disclosure provides methods of using the panels for the detection and quantification of CD3− cells in samples obtained at different stages of manufacturing of a T cell product for immunotherapy.

In one embodiment, the disclosure provides methods that may be used to identify, quantify, and optionally isolate, a variety of specific cell types using their cell surface marker pattern. These include, but are not limited to leukocytes, T cells, natural killer (NK) cells, natural killer T-cells (NKT cells), monocytes, B cells, early B progenitor cells, and stem cells. In one embodiment, the presence or absence of seven or more cell surface markers is determined simultaneously. In one embodiment, fluorescence activated cell sorting (FACS) analysis may be carried out all at once on a population of cells and it is possible to determine all at once what cells are present or absent based on their cell surface markers. In one embodiment, the method further assesses the cells' viability simultaneously with the cell surface markers. In one embodiment, it is not necessary to run the FACS analysis more than once or with multiple samples in order to be able to characterize the cell impurities in a T cell product.

In one embodiment, the method provides for the detection and/or quantification of the total amount of T lymphocytes in a sample. In one embodiment, the method provides for the detection and quantification of the total amount of non-T lymphocytes in the same sample.

In one embodiment, the sample is a blood sample from either a healthy donor or a patient (e.g., a cancer patient). In one embodiment, the sample is an apheresis sample. In one embodiment, the sample is from bone marrow. In one embodiment, the sample is a commerically avaiable mixture of blood cells such as as CYTO-TROL, Stem-Trol, Pan T cells, CD56+NK cells, ALL patient's apheresis, NHL'patients apheresis among others. In one embodiment, the sample is obtained from the manufacturing of a T cell product for immunotherapy. In one embodiment, the T cell product is a chimeric antigen receptor (CAR)-T cell product. In one embodiment, the sample is obtained after enrichment of the apheresis product in T cells by density gradient separation. In one embodiment, the sample has been obtained after enrichment ofthe apheresis product in CD4+ and/or CD8+ T cells by magnetic bead cell separation. In one embodiment, the sample is obtained from the end product ready for administration for immunotherapy.

In one embodiment, the method provides for the the detection and quantification of the specific combination of cell populations identified in Table 2, or subcombinations thereof (at least two, at least three, at least four, at least 5, at least 6, at least 7). In one embodiment, the specific combination or subcombination of cells is identified by the specific combination or subcombination (e.g., CD45, CD10, CD19) ofmarkers described in Table 2. In one embodiment, these markers are further combined with CD8 and CD4. Note that there are other possible cell surface markers that may be used to characterize “contaminating” cells in otherwise enriched lymphocyte compositions (e.g., CD25, CD2, CD7, and CD5). In one embodiment, the disclosure further provides a method to more specifically identify various types of cancer cells that may be present in a T cell population, where the T cell population is obtaining from an Acute Lymphocytic Leukemia (ALL) or Non-Hodgin Lymphoma (NL) patient. In one embodiment, Table 2 shows an exemplary specific combination of markers that is described in this application.

TABLE 2A
Exemplary Selection of Cell Surface Markers and Associated Parameters

| Antigen | Reporting Parameter Phenotype | Key Reporting Parameter/Unit |
|---|---|---|
| CD45 | Live/Singlet/Total Leukocyte/CD45⁺ | % CD45⁺ of Total Leukocytes |
| | Live/Singlet/Total Leukocyte/CD45^{dim} | % CD45^{dim} of Total Leukocytes |
| CD3 | Live/Singlet/Total Leukocyte/CD45⁺/CD14⁻/CD56⁻/ | % CD3⁺ T cells of Total Leukocytes |
| | CD3⁺ | |
| | Live/Singlet/Total Leukocyte/CD45⁺/CD14⁻/CD3⁻ | % CD3⁻ non-T cells of Total Leukocytes |
| CD56 | Live/Singlet/Total Leukocyte/CD45⁺/CD14⁻/ | % CD56⁺CD3⁺ NKT cells of Total |
| | CD3⁺CD56⁺ | Leukocytes |
| | Live/Singlet/Total Leukocyte/CD45⁺/CD14⁻/CD3⁻/ | % CD56⁺CD3⁻ NK cells of Total |
| | CD56⁺ | Leukocytes |
| CD14 | Live/Singlet/Total Leukocyte/CD45⁺/CD14⁺ | % CD14⁺ monocyte of Total Leukocytes |
| | Live/Singlet/Total Leukocyte/CD45⁺/CD14⁺CD56⁺ | % CD14⁺ CD56⁺ cells of Total Leukocytes |
| CD19 | Live/Singlet/Total Leukocyte/CD45⁺/CD14−/CD3− | % CD19⁺ B cells of Total Leukocytes |
| | CD56−/CD19⁺ | |
| | Live/Singlet/Total Leukocyte/CD45⁺/CD14⁻/CD3⁻ | % CD19⁺ CD34⁺ B progenitor cells of |
| | CD56⁻/CD19⁺CD34⁺ | Total Leukocytes |
| | | % CD19⁺ CD34⁺ B cells of CD3⁻ |
| CD34 | Live/Singlet/Total Leukocyte/CD45⁺/CD14⁻/CD3⁻ | % CD34⁺CD19⁺ of Total Leukocytes |
| | CD56⁻/CD19⁻/CD34⁺ | % CD34⁺CD19⁺ of CD45^{dim} |
| | Live/Singlet/Total Leukocyte/CD45+/CD14−/CD3− | % CD34⁺CD19⁺ B progenitor cells of |
| | CD56⁻/CD19⁺ CD34⁺ | Total Leukocytes |
| | | % CD34⁺CD19⁺ B progenitor cells of CD3⁻ |
| | Live/Singlet/Total Leukocyte/CD45^{dim}/CD34⁺CD19⁺ | % CD34⁺CD19⁺ of Total Leukocytes |
| | | % CD34⁺CD19⁺ of CD45^{dim} |
| | Live/Singlet/Total Leukocyte/CD45^{dim}/CD34⁺CD10⁺ | % CD34⁺CD10⁺ of Total Leukocytes |
| | | % CD34⁺CD10⁺ of CD45^{dim} |
| CD10 | Live/Singlet/Total Leukocyte/CD45^{dim}/CD10⁺CD19⁺ | % CD10⁺CD19⁺ of Total Leukocytes |
| | | % CD10⁺CD19⁺ of CD45^{dim} |

⁺means the cells display detectable levels of the marker.
⁻means the cells do not display detectable levels of the marker.
^{dim}means the cells display dim levels of the marker.

TABLE 2B
Exemplary Selection of
Cell Surface Markers and Associated Parameters

| | | Fluorophore/ | |
|---|---|---|---|
| Antibody | Clone | Conjugate | Purpose |
| CD3 | SK7 | APC | Pan T cell marker |
| CD10 | HI10a | FITC | Common ALL antigen; early |
| | | | B progenitor cell marker |
| CD14 | MϕP9 | PerCP-Cy5.5 | Monocyte marker |
| CD19 | HIB19 | PE-Cy7 | B cell marker |
| CD34 | 561 | BV421 | Stem and progenitor cell |
| | | | marker |
| CD56 | NCAM16.2 | PE | NK cell marker |
| CD45 | HI30 | V500 | Pan leukocyte marker |
| Live/ | NA | APC-CY7/ | Cell Viability |
| Dead Dye | | Near-IR dye | |

In one embodiment, CD4+ T cells are identified as CD3+CD4+CD45+ cells. In one embodiment, CD8+ T cells are identified as CD3+CD8+CD45+ cells. In one embodiment, CD45 is used for the detection of CD45+ leukocytes as well as to differentiate CD45dim B3-blasts from CD45+ population. In one embodiment, CD3 is used to differentiate CD3+ T cells from CD3− non-T cells. In one embodiment, CD56 is used to differentiate CD56+CD3+ NIK T cells and CD56+CD3− NK cells. In one embodiment, CD14 is used to identify general CD14+ monocytes and aberrant cells co-expressing CD56 and/or CD34 antigen. In one embodiment, CD34 is used to differentiate immobilized CD34+ cells in periphery, CD34+CD19+ and CD19-B-blast cells. In one embodiment, CD19 is used to differentiate normal and aberrant CD19+ B cells expressing CD34 and/or CD10 surface antigen. In one embodiment, CD10 is used to differentiate aberrant CD19+ early stage B progenitor cells or CD10+ immature B cells. In one embodiment, CD56+CD3- and CD56+CD3+ cells are generally defined as NK and NKT cells respectively as CD56 antigen is traditionally considered a NK cell marker in the hematopoietic system. However, it is worth noticing that CD56 expression has been reported to be not limited to NK or NKT cells, but also on other blood cells such as 76 T cells, ap T cells and dendritic cells.

In one embodiment, the disclosure provides a method wherein each of these markers is recognized by an antibody that is fluorescently labeled with a different fluorochrome. In one embodiment, the antibody is a polyclonal antibody. In one embodiment, the antibody is a monoclonal antibody.

Multicolour flow cytometry, as opposed to single-colour flow cytometry, introduces a higher technical difficulty in assay development. To analyse several surface markers simultaneously, each surface marker requires a specific antibody for detection. In flow cytometry it is best to use antibodies directly conjugated to fluorochromes instead of primary antibodies for detection and secondary antibodies for signal amplification. Therefore, when using multiple antibodies simultaneously, their conjugated fluorochromes must be chosen wisely so that they do not overlap in their emitted wavelengths. Fluorochromes that are as far apart as possible in the colour spectra may be chosen. Panel selection depends on multiple factors including accurate compensation and antigen-fluorochrome balancing.

In one embodiment, each antibody is labeled with a different fluorochrome/fluorophore. In one embodiment, the fluorochromes may be selected from any fluorochrome known in the art based on, for example, the relative abundance of the cell surface marker on the surface of the cells and the relative fraction of the cell population that each cell type represents.

In one embodiment, the fluorophore brightness increases in the order V500, near-IR dye (lowest); APC-Cy7, PerCP-Cy5.5; FITC; PE-Cy7; BV421, APC; PE, PE-Cy7 (highest). In one embodiment, the antigen abundance and/or density decreases in the order of CD45+(highest); CD3+; CD14+, CD19+; CD56+; CD10+; and CD34+(lowest). Control purified Pan-T cells, human peripheral blood CD19+ B cells, human peripheral blood NK cells, and other purified cells are available in the art from different manufacturers (e.g., StemCell Technologies).

Strategically, antigens in higher abundance are matched with dimmer flurochromes whereas those antigens with low abundance are matched with brighter fluorochroms. There are various industry standards known to one of ordinary skill in the art.

In one embodiment, the fluorochromes may be selected from any fluorochrome, including V500 (or any other blue emission dye), FITC (or any other green emission dye), BV421 (or any other blue emission dye), PE (or any other yellow emission dye), APC (or any other red emission dye), PE-Cy7 (or any other far red emission dye), PerCP.Cy5.5 (or any other far red emission dye), PacificBlue (or any other blue emission dye), PerCP (or any other red emission dye, any AlexaFluor (e.g., AlexaFluor700 (or any other red emission dye), AlexaFluor647 (or any other red emission dye), V450 (eg., BD Horizon V450, or any other blue emission dye), APC-Cy7 (or any othe infrared emission dye), SAV-TR-PE, PE-Cy7 (or any other infrared emission die), PE-Texas Red, Texas Red (or any other orange emission dye), AmCyan (or any other green emission dye), Alexa Fluor 488 (or any other green emission dye), PE-Cy5 (or any other red emission dye), DyeCycle dyes, Fluo-3, Fluo-5, Fura dyes, Qdot dyes, FVS dyes, Sytox dyes, and any other fluorescent dyes available in the art. In one embodiment, the live/dead dye is APC-CY7/Near-IR dye.

In one embodiment, one or more of the fluorescently-labeled antibodies is selected from the antibodies in Table 3.

TABLE 3
Exemplary Fit-for-Purpose Antibody Panels

| Antigen | CD45 | CD10 | CD34 | CD56 | CD3 | CD19 | CD14 |
|---|---|---|---|---|---|---|---|
| Exemplary | V500 | FITC | BV421 | PE | APC | PE-Cy7 | PerCP.Cy5.5 |
| Fluorochrome | | | | | | | |
| Other | ANY | ANY | ANY | ANY | ANY | ANY | ANY |
| Fluorochromes | | | | | | | |

In one embodiment, the fluorochromes are distributed differently than in the specific allocation in Table 3. For example, in one embodiment, the anti-CD45 antibody is FITC-labeled and the anti-CD10 antibody is V500 labeled. In one embodiment, at least one of the antibody labels is selected from other fluorescent labels available in the art. In one embodiment, at least one of the antibodies that is used to identify the cells in the sample is not from Table 3.

In one embodiment, each of the anti-CD45, anti-CD10, anti-CD34, anti-CD56, anti-CD3, anti-CD19, and anti-CD14 antibodies may be custom made. In one embodiment, any of these antibodies may be selected from any commercially available antibody against these cell surface markers. There are numeours commercially available antibodies against these marker antibodies, which may be acquired from, for example, BD Biosciences, Abcam, Thermofisher, Sinobiological, Biolegend, R&D Systems, Sigma Aldrich, Stem Cell, Santa Cruz Biotechonologies, ProteinTech, or any other antibody provider. In one embodiment, one or more antibodies is selected from the antibodies in Table 4.

TABLE 4
Exemplary Clones for a fit-for-purpose panel.

| Antigen | CD45 | CD10 | CD34 | CD56 | CD3 | CD19 | CD14 |
|---|---|---|---|---|---|---|---|
| Exemplary | H130 | H10a | 561 | NCAM16.2 | SK7 | HIB19 | MϕP9 |
| Choice | | | | | | | |
| Other Clones | ANY | ANY | ANY | ANY | ANY | ANY | ANY |

In one embodiment, the anti-CD19 antibody is selected from clones SJ25C1 and HIB19. In one embodiment, the anti-CD14 antibody is selected from clones MϕP9 and M5E2. In one embodiment, the anti-CD56 antibody is selected from clones NCAM16.2 and HCD56. In some embodiments, the specificity for CD34, CD19, and CD56 conjugated antibodies may be examined by testing known positive and negative samples for the corresponding markers. In one example, for CD34 antibody specificity, Stem-Trol (commercially sourced/manufactured CD34+ positive control cells) from StemCell Technologies may be used as the positive sample. In some embodiments, MAVER-1/MRL3008 (CD19+ B cell line), and pure NK cells (from StemCell Technologies) may be used as positive samples for the specificity of CD19 and CD56 antibodies, respectively. CD34+ cells, CD19+ cells and NK cells percentages are the output measurements for this assessment. The positive control testing material, CYTO-TROL, may also used in the specificity test, as it has lot-specific reference ranges provided by the manufacturer. In one embodiment, the accuracy and other performance parameters of a method that uses one or more antibodies other than those in Tables 4-6 may be assessed by using the methods described in the EXAMPLES as reference values. The linearity of each assay may be determined using serial dilutions as per established methods.

In one embodiment, one or more antibodies is selected from the antibodies in Table 5. More details regarding the source of these particular clones may be found in the EXAMPLES.

TABLE 5
Exemplary clone/flurochrome combination for a fit-for-purpose panel.

| Antigen | CD45 | CD10 | CD34 | CD56 | CD3 | CD19 | CD14 |
|---|---|---|---|---|---|---|---|
| Clone | HI30 | H10a | 561 | NCAM16.2 | SK7 | HIB19 | MϕP9 |
| Fluorochrome | V500 | FITC | BV421 | PE | APC | PE-Cy7 | PerCP.Cy5.5 |

Panel selection also depends on optimally titrated antibodies. In one embodiment, one or more of the antibodies and their respective amounts in a staining composition are selected from those of Table 6. In one embodiment, the composition, also described herein as an antibody cocktail, has been lyophilized.

TABLE 6
Exemplary Amounts for an exemplary fit-for-purpose panel for 1 × 10⁶ cells.

| Antigen | CD45 | CD10 | CD34 | CD56 | CD3 | CD19 | CD14 |
|---|---|---|---|---|---|---|---|
| Clone | HI30 | H10a | 561 | NCAM16.2 | SK7 | HIB19 | MϕP9 |
| Fluorochrome | V500 | FITC | BV421 | PE | APC | PE-Cy7 | PerCP.Cy5.5 |
| Antibody | 0.20 | 0.52 | 0.5 | 0.02 | 0.05 | 0.05 | 0.065 |
| μg/Test | | | | | | | |
| Antibody/ | 2.0 μL | 1.3 μL | 5.0 μL | 1.3 μL | 1.0 μL | 1.0 μL | 1.3 μL |
| μl/Test* | | | | | | | |

*total 12.9/100 μl, plus, optionally, 87.1 μl of staining buffer and 200 μl of LIVE/DEAD Near-IR Fixable Dye at 1:3000 dilution.

In one embodiment, the total amounts of each antibody is different from those in Table 6. In one embodiment, the ratio of each antibody in the fit-for-purpose product is as reflected in Table 7.

TABLE 7
Exemplary ratios (%) of antibody in the staining composition/product,
relative to the total amount of antibody in the product.

| Antigen | CD45 | CD10 | CD34 | CD56 | CD3 | CD19 | CD14 |
|---|---|---|---|---|---|---|---|
| Clone | HI30 or | H10a | 561 or | NCAM16.2 | SK7 | HIB19 | MϕP9 or |
| | other | or | other | or other | or | or other | other |
| | | other | | | other | | |
| Fluorochrome | V500 or | FITC | BV421 | PE or other | APC | PE-Cy7 | PerCP.Cy5.5 |
| | other | or | or other | | or | or other | or other |
| | | other | | | other | | |
| Antibody | About | About | About | About | About | About | About 0.5% |
| (% total**) | 1.6% | 4.0% | 4.0% | 0.16% | .41% | .41% | |
| Antibody | About | About | About | About | About | About | About |
| (% total^{##}) | 8.5% | 34.0% | 32.7% | 1.3% | 3.3% | 3.3% | 17.0% |
| Antibody | About | About | About | About | About | About | About |
| (% total^{%%}) | 8.5% | 34.0% | 32.7% | 1.3% | 3.3% | 3.3% | 17.0% |
| Antibody | 8.5% | 34.0% | 32.7% | 1.3% | 3.3% | 3.3% | 17.0% |
| (% total) | | | | | | | |

**“about” means within 1 standard of deviation
^{##}“about” means plus or minus 10% of the recited number
^{%%}“about” means plus or minus 20% of the recited number

In one embodiment, one or more of the antibodies is present in an amount that is 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, etc., or fractions thereof more than or less that the amounts in Table 6. In one embodiment, the ratio of CD10, CD34, CD56, CD3, CD19, and/or CD14 antibody changes in the staining composition/product/cocktail, relative to the amount of CD45 antibody. In one embodiment, the ratio of CD45, CD34, CD56, CD3, CD19, and/or CD14 antibody changes, relative to the amount of CD10 antibody. In one embodiment, the ratio of CD45, CD10, CD56, CD3, CD19, and/or CD14 antibody changes, relative to the amount of CD34 antibody; and so on and so forth. In one embodiment, the ratios change because the fluorochrome changes thereby changing the number of moles of antibody per microgram relative to those of Table 6. In one embodiment, the fluorochrome changes but the ratio of antibodies in terms of moles of unlabeled antibody is the same as that in Table 6.

In one embodiment, the total amount of antibody per test is that in Table 6. In one embodiment, the amount of each individual antibody per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, or 500 percent, or fractions thereof, relative to the amounts in Table 6. In one embodiment, the amount of each individual antibody per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, or 500 fold, or fractions thereof, relative to the amounts in Table 6.

In one embodiment, the amount of each of the antibodies per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, 500, 600, 700, 800, 900, 1000 nanograms, or fractions thereof, relative to the amounds in Table 6.

In one embodiment, the amount of each of the antibodies per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, 500, 600, 700, 800, 900, 1000 micrograms, or fractions thereof, relative to the amounts in Table 6.

In one embodiment, to optimize the antibody panels, antibodies may be titrated to determine the use volume/concentration that gives a robust signal-to-noise ratio, minimum background, and staining intensity with consisten percentage positive signal. In one embodiment, in order to determine the optimal concentration for the staining, antibodies may be serially diluted and the stain index (SI) calculated as [MFIp-MFIn]/2×rSDn, where MFIp is median fluorescence intensity (MFI) for the positive population, MFIn is MFI for the negative population, and rSDn is robust standard deviation of the negative population. In one embodiment, this is done by a method described in Maecker H T. et al. Cytometry Part A 2006 (69A): 1037-1042. In one embodiment, a plot of SI may be created to select the robust mass of antibodies that gives significant SI values. Excess antibody volume may artificially increase both the positive and negative signal of the entire cell population.

In one embodiment, fewer than all seven antibodies described in the Tables above are used in the method of identifying CD3− impurities in a T cell sample and/or are mixed in the staining composition or cocktail. In one embodiment, the cocktail comprises only antibodies to detect CD45+CD3+ lymphocytes (all lymphocytes in a mixture). In one embodiment, the cocktail comprises only antibodies to detect NK T cells, which are CD45+/CD3+/CD56+. In one embodiment, the cocktail comprises only antibody to detect NK cells, which are CD45+/CD3−/CD56+. In one embodiment, the cocktail comprises only antibodies to detect monocytes, which are CD45+/CD3−/CD14+CD19−. In one embodiment, the cocktail comprises only antibodies to detect B cells, which are CD45+/CD3−/CD14−CD19+. In one embodiment, the cocktail comprises only antibodies to detect stem and progenitor cells, which are CD45+/CD34+. In one embodiment, the cocktail comprises only antibodies to detect early B progenitor cells, which are CD45dim/CD10+CD19+. In some embodiments, the cocktail comprises antibodies for any combination thereof. In some embodiments, the cocktail is lyophilized.

In one embodiment, the antibody cocktail composition comprises enough antibodies for a pre-determined number of tests (each test being the contacting of a population of cells with the cockatil of antibodies). In one embodiment, the total volume of antibody cocktail/mixture per test sample is 100 μL. In one embodiment, the total volume of antibody cocktail/mixture per test sample is 10 μL, 50 μL, 100 μL, 200 μL, 300 μL, 400 μL, 500 μL, 600 μL, 700 μL, 800 μL, 900 μL, or 1000 μL. In one embodiment, the total volume of antibody cocktail/mixture per test sample is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, or 100 μL.

In one embodiment, each test is designed to analyse approximately 1 million blood cells. In one embodiment, each test is designed to analyse approximately 2 million, 3 million, 4 million, 5 million, 6 million, 7 million, 8 million, 9 million, or 10 million cells. In one embodiment, the cell sample has a volume of approximately 200 μL. In one embodiment, the cell sample has a volume of approximately 10 μL, 50 μL, 100 μL, 200 μL, 300 μL, 400 μL, 500 μL, 600 μL, 700 μL, 800 μL, 900 μL, or 1000 μL. In one embodiment, the cell sample comprises 1 million cells in 200 μL of cell staining buffer. In one embodiment, each sample comprises approximately 1 million cells in 200 μL of cell stain buffer and this may be mixed with 100 μL of antibody mixture for analysis.

In one embodiment, the disclosure provides a container carrying enough of a cocktail/mixture of the seven antibodies of the above tables for 20 samples. In one embodiment, the container carries enough antibody mixture/cocktail for staining 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, or 100 samples per container. In one embodiment, the mixture/cocktail is lyophilized. In one embodiment, the mixture/cocktail is suspended in a buffer.

In one embodiment, the lyophilized cocktail (for example, the amounts specified in Table 6 or Table 7) is resuspended in a buffer appropriate for use in FACS. In one embodiment, the resuspension is stable for at least 10 days at room temperature, when resuspended in 2000 μL of buffer. In one embodiment, the resuspension is stable for at least 3 months at room temperature, when resuspended in 400 μL of buffer. In one embodiment, the resuspension is stable for at least or approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, or 100 days at room temperature when resuspended in 100, 200, 300, 400, 500, 600, 700, 800, 900, 1000, 1100, 1200, 1300, 1400, 1500, 1600, 1700, 1800, 1900, or 2000 μL of buffer.

In one embodiment, the assay as a lower limit of quantitation (LLOQ) of each of the CD3− populations (e.g., CD34+, CD56+NK, CD19+ B cells) of about 0.2% for CD34+ cells and CD19+ B cells and about 1.4% for CD56+CD3− NK cells. In one embodiment, the LLOQ is about 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50%. In one embodiment, the LLOQ may be assessed as a linearity study by mixing target population with a negative population. Serial dilutions may be made by a factor of 2 (6.25%, 3.13%, 1.56%, 0.78%, 0.39%, 0.2%, 0.1%, 0.05%, 0.02%, 0.01% and 0.00%), and each dilution may be tested in triplicate. The lowest dilution with the acceptable % Recovery (within 80% to 120%) and the acceptable % CV for replicates (≤25) may be set as the LLOQ. In one embodiment, a LLOQ test may be performed to confirm if the assay is sensitive enough to detect CD34+ populations below 10%. CD34+ cells are typically rare in human PBMCs.

In one embodiment, the sample is an apheresis sample comprising healthy donor PBMC. The typical cellular composition of such sample comprises 25-60% CD4+ T cells, 5-30% CD8+ T cells, 5-10% CD19+ B cells, 10-30% CD56+CD3− NK cells, and 4-10% CD14+ monocytes. In one embodiment, the sample is an apheresis sample comprising PBMC from a cancer patient.

In one embodiment, the disclosure provides a method of characterizing CD3-cells (e.g., NK-T cells, NK cells, monocytes, early B cell progenitor cell, or combinations thereof), which may be considered impurities, in a T cell preparation comprising contacting a sample of the T cell preparation with a cockatil of antibodies as described in this disclosure and analyzing the mixture for the distribution of cells with specific cell surface markers by fluorescence detection methods.

In one embodiment, the disclosure provides a method of treating cancer in a subject in need thereof with a T cell preparation wherein one or more of the CD3− impurities (e.g., NK-T cells, NK cells, monocytes, early B cell progenitor cell, or combinations thereof) in the T cell preparation have been or are characterized by a method that requires the use of one or a mixture/cocktail of antibodies as described in this disclosure. In one embodiment, the T cell preparation is autologous. In one embodiment, the T cell preparation is allogeneic. Examples of T cell populations and of methods of preparation of exemplary T cell populations for immunotherapy are described earlier in this disclosure. In one embodiment, the T cells are engineered with a CAR or T cell receptor. Examples of CARs and T cell receptors are described earlier in this disclosure.

In one embodiment, the disclosure provides a method for determining whether a T cell product is suitable for immunotherapy, comprising characterizing one or more of the CD3− cell impurities (e.g., NK-T cells, NK cells, monocytes, early B cell progenitor cell, or combinations thereof) in the T cell product using one of the antibodies or cocktail of antibodies described in this disclosure and determining whether the T cell product is suitable based on the levels of CD3− cell impurities in the T cell product. In one embodiment, the acceptable levels are set by regulatory authorities (e.g., FDA, EMEA, etc). In some embodiments, the levels of at least one of the cell types is above accepted levels. In some embodiments, the levels of at least one of the cell types is below accepted levels.

In one embodiment, the disclosure provides a method/assay or a kit for identifying at least one of leukocytes, NK-T cells, NK cells, monocytes total lymphocytes, early B cell progenitor cell, or combinations thereof in blood cell populations. In some embodiments, the assay and/or kit is used to characterize CD3− cells in T cell products for immunotherapy. In one embodiment, the kit comprises (a) one of more antibodies to detect one or more cell markers for any one or more of these cells (see, e.g., Table 2) and (2) reagents to carry on the binding of the antibody with the cell surface markers, and, optionally, (3) instructions for using the reagents for the kit's purpose. In some embodiments, the antibodies (or or more) are all lyophilized together in the same container (e.g., a Lyovial). In one embodiment, the antibodies are selected from Table 3. In one embodiment, the antibodies are selected from Table 4. In one embodiment, the antibodies are selected from Table 5. The amounts of each antibody in the vial(s) of the kit may vary from these amounts, as described elsewhere in the specification.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes may be made without departing from the spirit and scope of the disclosure.

### EXAMPLES

### Example 1

### Design of a Fit-for-Purpose Flow Cytometry Panel for CD3− Impurities

A fit-for-purpose 8 color T-cell impurity flow cytometry panel was experimentally developed to assess CD3+ cell purity in T cell samples or products, together with viable cells. The panel may be used in characterizing CD3− impurities (NK-T cells, NK cells, B cells, monocytes) in blood cell samples, including those obtained by apheresis, PBMCs, and those prepared throughout the manufacturing of T cell products for immunotherapy.

A panel of cell surface markers was first selected to identify the different cell populations in the blood samples. The markers were as shown in Table 8.

TABLE 8
Markers used and their associated reporting parameters.

| Antigen | Reporting Parameter Phenotype | Key Reporting Parameter/Unit |
|---|---|---|
| CD45 | Live/Singlet/Total Leukocyte/CD45⁺ | % CD45⁺ of Total Leukocytes |
| | Live/Singlet/Total Leukocyte/CD45^{dim} | % CD45^{dim} of Total Leukocytes |
| CD3 | Live/Singlet/Total Leukocyte/CD45⁺/CD14⁻/CD56⁻/ | % CD3⁺ T cells of Total Leukocytes |
| | CD3⁺ | |
| | Live/Singlet/Total Leukocyte/CD45⁺/CD14⁻/CD3⁻ | % CD3⁻ non-T cells of Total Leukocytes |
| CD56 | Live/Singlet/Total Leukocyte/CD45⁺/CD14⁻/ | % CD56⁺CD3⁺ NKT cells of Total |
| | CD3⁺CD56⁺ | Leukocytes |
| | Live/Singlet/Total Leukocyte/CD45⁺/CD14⁻/CD3⁻/ | % CD56⁺CD3⁻ NK cells of Total |
| | CD56⁺ | Leukocytes |
| CD14 | Live/Singlet/Total Leukocyte/CD45⁺/CD14⁺ | % CD14⁺ monocyte of Total Leukocytes |
| | Live/Singlet/Total Leukocyte/CD45⁺/CD14⁺CD56⁺ | % CD14⁺ CD56⁺ cells of Total Leukocytes |
| CD19 | Live/Singlet/Total Leukocyte/CD45⁺/CD14−/CD3− | % CD19⁺ B cells of Total Leukocytes |
| | CD56−/CD19⁺ | |
| | Live/Singlet/Total Leukocyte/CD45⁺/CD14⁻/CD3⁻ | % CD19⁺ CD34⁺ B progenitor cells of |
| | CD56⁻/CD19⁺CD34⁺ | Total Leukocytes |
| | | % CD19⁺ CD34⁺ B cells of CD3⁻ |
| CD34 | Live/Singlet/Total Leukocyte/CD45⁺/CD14⁻/CD3⁻ | % CD34⁺CD19⁺ of Total Leukocytes |
| | CD56⁻/CD19⁻/CD34⁺ | % CD34⁺CD19⁺ of CD45^{dim} |
| | Live/Singlet/Total Leukocyte/CD45+/CD14−/CD3− | % CD34⁺CD19⁺ B progenitor cells of |
| | CD56⁻/CD19⁺ CD34⁺ | Total Leukocytes |
| | | % CD34⁺CD19⁺ B progenitor cells of CD3⁻ |
| | Live/Singlet/Total Leukocyte/CD45^{dim}/CD34⁺CD19⁺ | % CD34⁺CD19⁺ of Total Leukocytes |
| | | % CD34⁺CD19⁺ of CD45^{dim} |
| | Live/Singlet/Total Leukocyte/CD45^{dim}/CD34⁺CD10⁺ | % CD34⁺CD10⁺ of Total Leukocytes |
| | | % CD34⁺CD10⁺ of CD45^{dim} |
| CD10 | Live/Singlet/Total Leukocyte/CD45^{dim}/CD10⁺CD19⁺ | % CD10⁺CD19⁺ of Total Leukocytes |
| | | % CD10⁺CD19⁺ of CD45^{dim} |

A specific panel of seven antibody and fluorophore combinations (plus a far red viability dye) were then developed following a variety of criteria. Antigens with higher abundance were matched with dimmer fluorochromes whereas those antigens with low abundance were matched with brighter fluorochromes. This enabled more sensitive detection by the panel. Various antibody-to-fluorochrome combinations were evaluated to minimize spectral spillover into neighboring channels in the instrument and to achieve best resolution for all populations. A compensation setup also ensured the correction of spectral spillover. Selected panels are described in Tables 9.1, 9.2, 9.3, 9.4, and 9.5.

TABLE 9.1
Antibody Panel

| Antigen | CD45 | CD10 | CD34 | CD56 | CD3 | CD19 | CD14 |
|---|---|---|---|---|---|---|---|
| Clone | HI30 | H10a | 561 | NCAM16.2 | SK7 | HIB19 | MϕP9 |
| Fluorochrome | V500 | FITC | BV421 | PE | APC | PE-Cy7 | PerCP.Cy5.5 |
| Antibody | 0.20 | 0.52 | 0.5 | 0.02 | 0.05 | 0.05 | 0.065 |
| μg/Test | | | | | | | |

TABLE 9.2
Isotype Antibody Panel

| | Antigen | | | | | | |
|---|---|---|---|---|---|---|---|
| | CD45 | IgG1 | IgG2a | IgG2b | CD3 | CD19 | CD14 |
| Clone | HI30 | MOPC-21 | MOPC-173 | MPC-11 | SK7 | HIB19 | MϕP9 |
| Fluorochrome | V500 | FITC | BV421 | PE | APC | PE-Cy7 | PerCP.Cy5.5 |
| Antibody μg/Test | 0.20 | 0.26 | 0.26 | 0.26 | 0.05 | 0.05 | 0.065 |

TABLE 9.3
Compensation Controls

| | Antigen | | | | | | |
|---|---|---|---|---|---|---|---|
| | CD45 | CD10 | CD34 | CD56 | CD3 | CD19 | CD14 |
| Clone | HI30 | H10a | 561 | NCAM16.2 | SK7 | HIB19 | MϕP9 |
| Fluorochrome | V500 | FITC | BV421 | PE | APC | PE-Cy7 | PerCP.Cy5.5 |
| Antibody μg/Test | 0.15 | 0.2 | 0.08 | 0.01 | 0.04 | 0.04 | 0.16 |

TABLE 9.4
SAMPLE ANTIBODY PANEL

| | Antigen | | | | | | |
|---|---|---|---|---|---|---|---|
| | CD45 | IgG1 | IgG2a | IgG2b | CD3 | CD19 | CD14 |
| Clone | HI30 | MOPC-21 | MOPC-173 | MPC-11 | SK7 | HIB19 | MoP9 |
| Fluorochrome | V500 | FITC | BV421 | PE | APC | PC7 | PerCP.Cy5.5 |
| Antibody μg/Test | 0.2 | 0.26 | 0.26 | 0.26 | 0.05 | 0.05 | 0.065 |

TABLE 9.5
ISOTYPE ANTIBODY PANEL

| | Antigen | | | | | | |
|---|---|---|---|---|---|---|---|
| | CD45 | IgG1 | IgG2a | IgG2b | CD3 | CD19 | CD14 |
| Clone | HI30 | MOPC-21 | MOPC-173 | MPC-11 | SK7 | HIB19 | MoP9 |
| Fluorochrome | V500 | FITC | BV421 | PE | APC | PC7 | PerCP.Cy5.5 |
| Antibody μg/Test | 0.2 | 0.26 | 0.26 | 0.26 | 0.05 | 0.05 | 0.065 |

To optimize the method panel, all seven antibodies were titrated to determine the use volume/concentration that gave a robust signal-to-noise ration, minimum background, staining intensity with consistent % positive signal. See Tables 9.1, 9.2, 9.3, 9.4 and 9.5.

The performance of selected antibody titration was verified by staining samples with antibody cocktail at the titrated volume/concentration, or at the manufacturer's recommended volume.

The reagents used for the method were as specified in Table 10.

TABLE 10
List of reagents used in method development.

| Reagent | Manufacturer |
|---|---|
| APC anti-human CD3 | Biolegend |
| FITC anti-human CD10 | Biolegend |
| PerCP-Cy5.5 anti-human CD14 | BD Biosciences |
| PE-Cy7 anti-human CD19 | BD Biosciences |
| BV421 anti-human CD34 | Biolegend |
| PE anti-human CD56 | BD Biosciences |
| BV421 mouse IgG2a | Biolegend |
| PE mouse IgG2b | Biolegend |
| FITC mouse IgG1 | Biolegend |
| V500 anti-human CD45 | BD Biosciences |
| LIVE/DEAD Fixable Near-IR | Thermo Fisher |
| Dead Cell Stain Kit | |
| Cytometer Set-up and | BD Biosciences |
| Tracking (CS&T) beads | |
| UltraComp eBeads Compensation | Thermo Fisher |
| Beads | Scientific |
| Cell Stain Buffer (BSA) | BD Biosciences |

In order to determine the optimal antibody concentration for the staining, antibodies were serially diluted and measured in duplicate. The stain index (SI) was calculated using the following equation:

$$Stain Index ( SI ) = \frac{[ {MFI}_{p} - {MFI}_{n} ]}{2 \times rSDn} ,$$

Where MFIp is median fluorescence intensity (MFI) for the positive population, MFIn is MFI for the negative population, and rSDn is robust standard deviation of the negative population.

A plot of SI was created to select the robust mass of antibodies that gives significant SI values. Excess antibody volume can artificially increase both the positive and negative signal of the entire cell population. Because the optimal antibody concentration cannot be determined by SI alone, the MFIs of the positive and negative target populations, as well as the frequency of positive population, were examined. For example, two different antibody clones were compared for the following antibodies and the clone with higher SI was selected to include in one of the exemplary panels:

PE-Cy7 CD19 antibody: Clones SJ25C1 and HIB19 were titrated and compared. Both clones showed similar specificity, but clone HIB19 had higher SI.

PerCP-Cy5.5 CD14 antibody: Clones MϕP9 and M5E2 were tested and showed similar specificity. Clone MϕP9 had higher SI.

PE CD56 antibody: Clones NCAM16.2 and HCD56 were titrated and compared. NCAM16.2 showed better specificity and resolution.

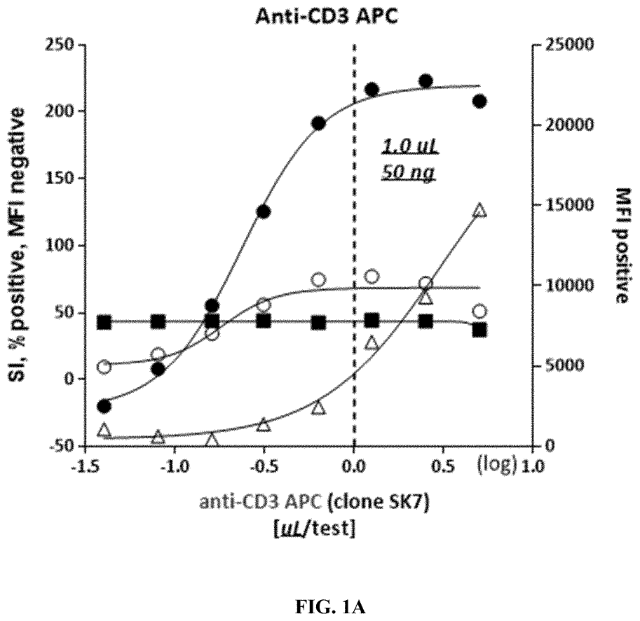

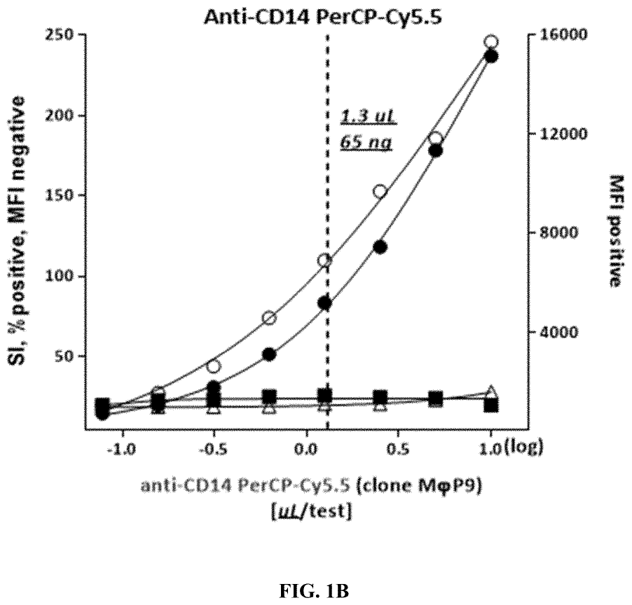

All of the antibodies used in the method were titrated during this optimization effort. As an example, the optimized antibody volume (μL) and concentration/mass (ng or μg) for anti-CD3 APC and anti-CD14 PerCP-Cy5.5 are indicated in FIGS. 1A and 1B, respectively. CD14 PerCP-Cy5.5 antibody showed no saturation of CD14+ MFI. A detailed examination of the flow plots revealed that 1.3 μL of antibody was sufficient to produce a positive signal and subsequently optimizes the log parameter for sample acquisition/analysis in FACSCanto II system. The antibody volumes per reaction for 1×10⁶ cells were finalized as in Table 12.

The performance of (A) a freshly made liquid cocktail of the above antibodies prepared fresh for each run from liquid stocks of each different antibody was compared with the performance of (B) a liquid cocktail of the same antibodies prepared fresh from a lyophilized cocktail of all seven antibodies, or used for several days after resuspension.

The (A) liquid cocktail and the (B) lyophilized cocktail were mixed from their individual components as described in Tables 11 and Table 12.

TABLE 11
Antibody amounts used in the preparation of a (A) liquid cocktail.

| | Antibody Volume/ |
|---|---|
| Antibody | Reaction (μL) |
| CD3-APC | 1.0 |
| CD14-PerCP-Cy5.5 | 1.3 |
| CD56-PE | 1.3 |
| CD19-PE-Cy7 | 1.0 |
| CD10-FITC | 1.3 |
| CD45-V500 | 2.0 |
| CD34-BV421 | 2.0 |
| Total antibody | 14.9 |
| BSA stain Buffer added | 85.1 |

TABLE 12
Antibody amounts used in the
preparation of one of the (B) lyophilized cocktails (lyovial).

| | Antibody Volume/ | Antibody |
|---|---|---|
| Antibody | Reaction (μL) | μg/Test |
| CD3-APC | 1.0 μL | 0.05 |
| CD14-PerCP-Cy5.5 | 1.3 μL | 0.065 |
| CD56-PE | 1.3 μL | 0.02 |
| CD19-PE-Cy7 | 1.0 μL | 0.05 |
| CD10-FITC | 1.3 μL | 0.52 |
| CD45-V500 | 2.0 μL | 0.20 |
| CD34-BV421 | 5.0 μL | 0.5 |
| Total antibody volume | 12.9 μL | |
| Staining Buffer (BSA) | 87.1 μL | |
| LIVE/DEAD Near-IR | 200 μL in | |
| Fixable Dye | 1:3000 dilution | |

An isotype control liquid mix was also prepared. Table 13.

TABLE 13
Antibody cocktails used in an Isotype mix.

| Antibody | Antibody Volume/Reaction (μL) |
|---|---|
| CD3-APC | 1.0 |
| CD14-PerCP-Cy5.5 | 1.3 |
| m IgG2b-PE | 1.3 |
| CD19-PE-Cy7 | 1.0 |
| CD45-V500 | 2.0 |
| m IgG1-FITC | 2.0 |
| m IgG2a-BV421 | 2.0 |
| Total antibody | 14.9 |
| BSA stain Buffer added | 85.1 |

A total of 100 μl of each cocktail was used per cell sample, which typically contains approximately 1 million cells. The (B) liquid cocktail prepared from the lyophilized combination of all seven antibodies was prepared by adding 2000 μl of BD BSA cell staining buffer added to the lyophilized antibody cocktail vial (Lyovial) and 100 μl of the resulting (B) liquid cocktail were also used per sample of approximately 1 million cells at the concentration of 5×10⁶ cells/mL of cell staining buffer). In general, the samples may be fresh apheresis samples, CD4+/CD8+ positive cells after positive selection of apheresis samples, or any other sample harvested throughtout the manufacturing of the T cell product, including the final CAR-T ot TCR-T cell harvest products. The lyovial contained enough antibodies for 20 tests. The amounts of each antibody were as shown in the Tables above.

### Example 2

### Staining Comparability of (A) Liquid and (B) Lyophilized Reagents

The reproducibility of the method was tested. Five healthy donor apheresis samples were tested in duplicates with two types of cocktails: (A) freshly made cocktail by combining individual antibodies in liquid format according to TABLE 5 and TABLE 6, or (B) lyophilized antibody cocktails (trial reagents). The tests were done in parallel, on the same day, by one analyst, to understand the comparability between lyophilized and liquid reagents and to assess the functionality of lyophilized reagents. The % change was determined for each parameter of interest, CD45+, T cells, NK cells, monocytes and B cells.

Apheresis samples were harvested from five healthy donors. A total of 200 microliters containing 1 million blood cells suspended in Cell Stain Buffer (BSA) were mixed with 100 microliters of either the (A) liquid cocktail or (B) the cocktail resuspended from a lyophilized cocktail. CYTO-TROL Control Cells ( )(Beckman Coulter), were used as positive controls. They are a lyophilized preparation of human lymphocytes that exhibit surface antigens detectable with the chosen monoclonal antibodies. These cells are isolated from peripheral blood and express antigens that are representative of those found on normal lymphocytes.

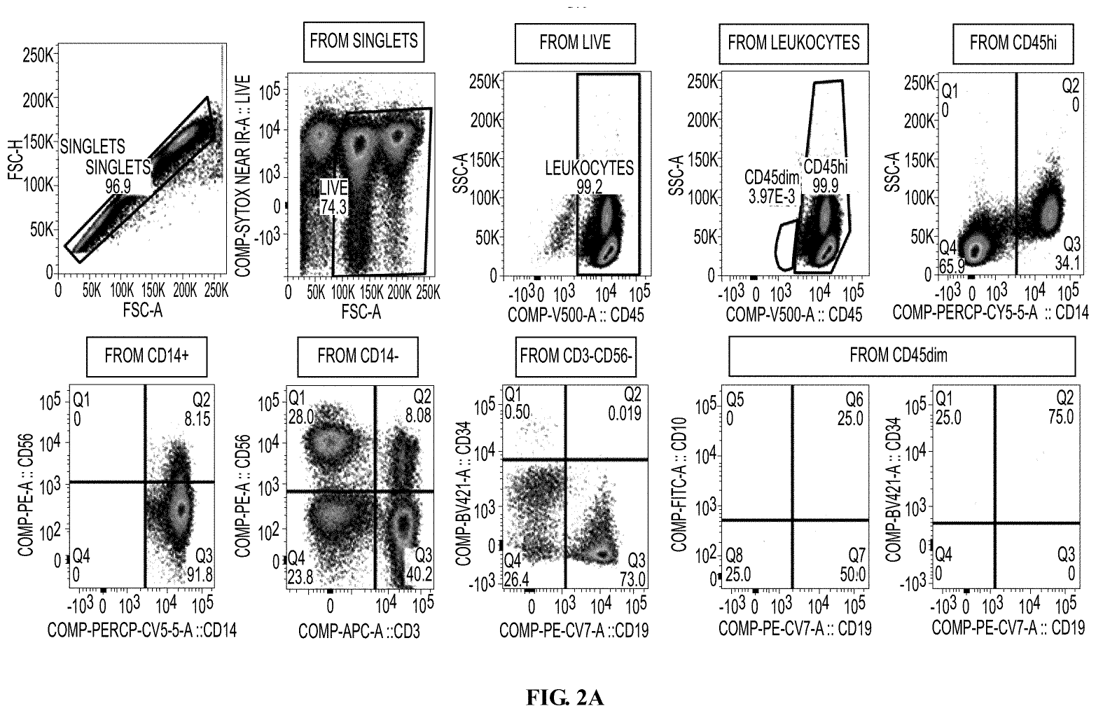

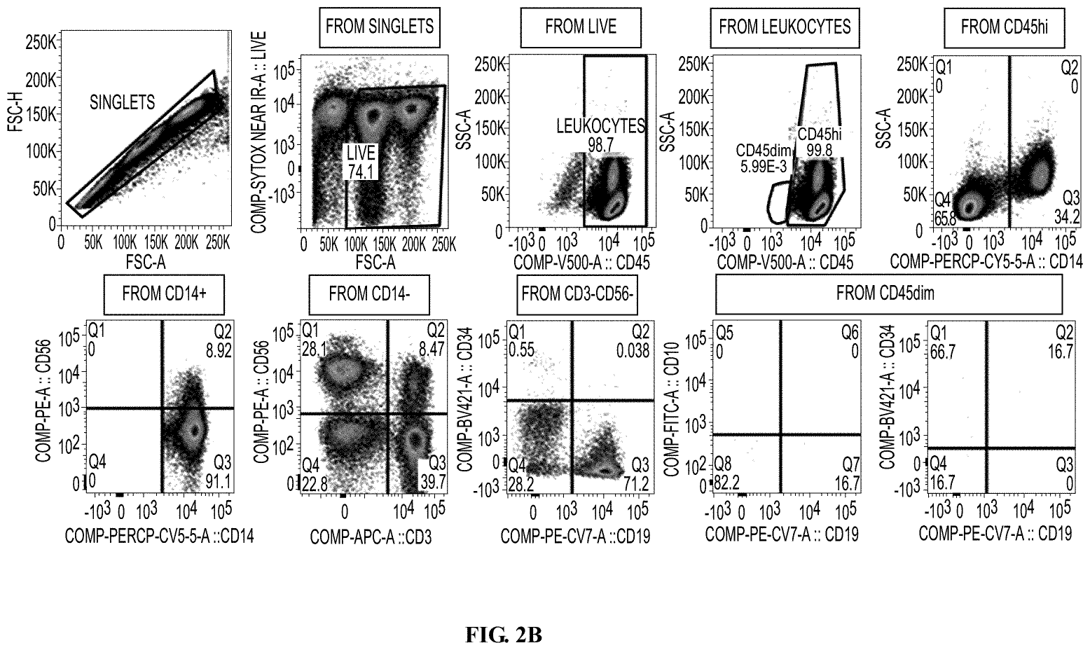

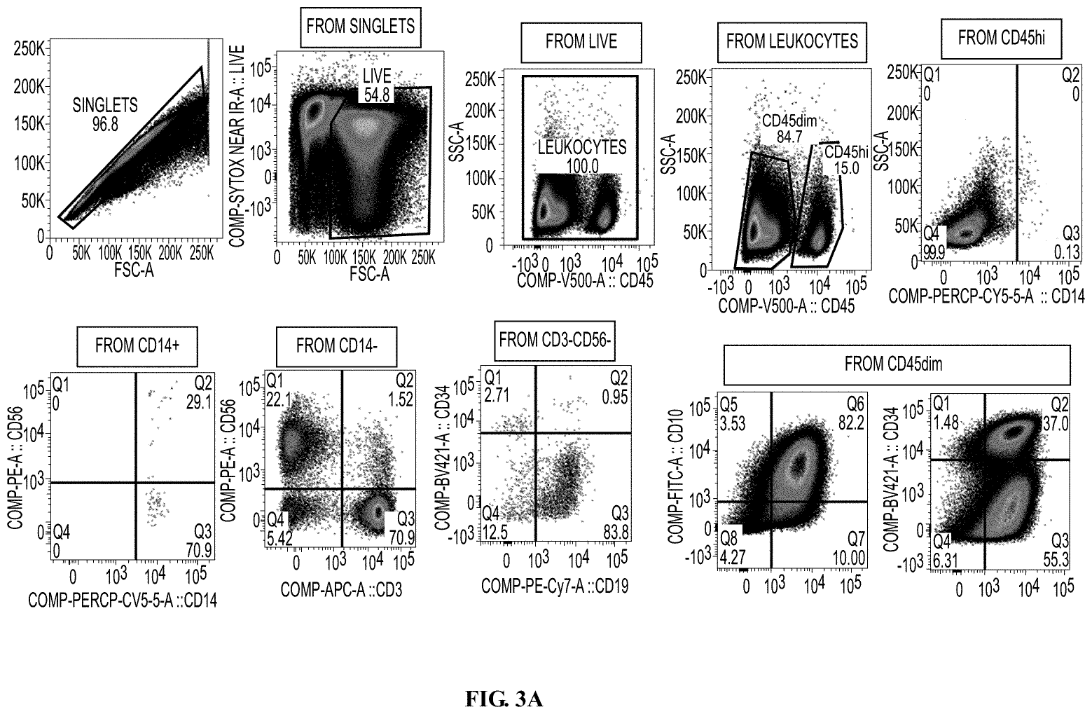

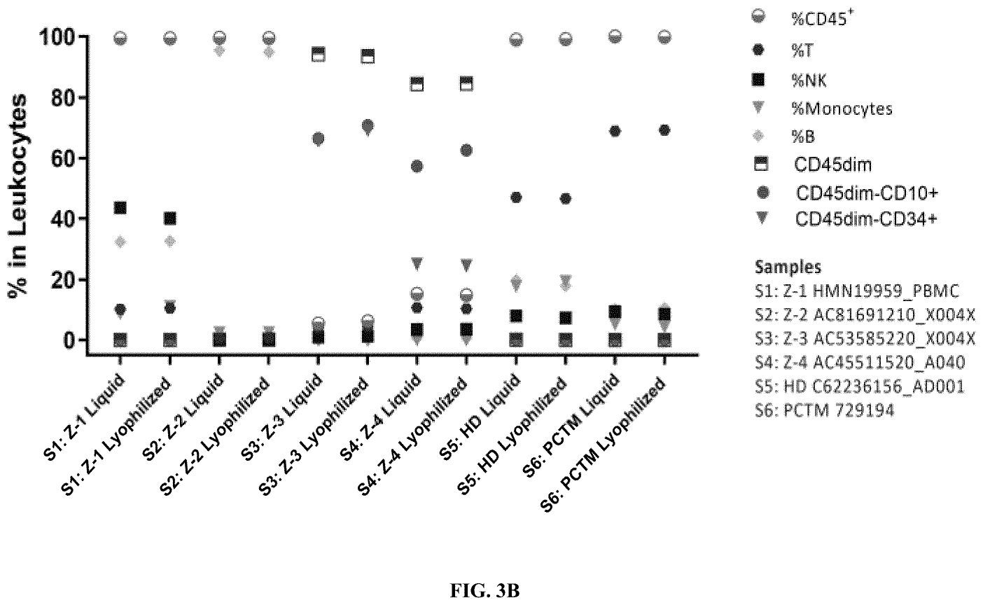

Sample were incubated at 2 to 8° C. for approximately 25 minutes protected from light. Once incubation was complete, 100 microliters of Cell Stain Buffer (BSA) were added to each sample and the samples centrifuged. The pelleted cells were washed three times with 200 microliters of Cell Stain Buffer (BSA) each and then stained with a viability dye. Samples were then processed by FACS, together with appropriate single-color compensation controls. The results are shown in FIGS. 2A and 2B (FIG. 3) and Table 14. The frequencies of CD3+ T cells and CD3− non-T cellular impurities were analyzed on a BD FACSCanto II Flow Cytometer using FlowJo software.

TABLE 14
Flow cytometric data of Staining comparison of Liquid and Lyophilized reagents.

| | | | % in CD45⁺ | | | |
|---|---|---|---|---|---|---|
| | | % CD45⁺ in | | NK | | |
| Reagent Type | Sample | Leukocytes | T cells | cells | Monocytes | B cells |
| Fresh liquid | S1 Apheresis | 98.8 | 51.6 | 9.7 | 14.6 | 20.6 |
| Lyophilized | | 98.5 | 48.5 | 9.1 | 17.9 | 19.9 |
| % Change | | 0.3 | 6.0 | 6.2 | −22.6 | 3.4 |
| Fresh liquid | S2 Apheresis | 98.5 | 58.9 | 13.6 | 11.9 | 11.8 |
| Lyophilized | | 98.3 | 58.2 | 13.4 | 11.3 | 12.2 |
| % Change | | 0.2 | 1.2 | 1.5 | 5.0 | −3.4 |
| Fresh liquid | S3 Apheresis | 99.2 | 66.2 | 8.8 | 10.8 | 10.7 |
| Lyophilized | | 99.2 | 66.9 | 8.7 | 10.6 | 11.4 |
| % Change | | 0.0 | −1.1 | 1.1 | 1.9 | −6.5 |
| Fresh liquid | S4 Apheresis | 97.9 | 54.2 | 10.2 | 21.3 | 5.2 |
| Lyophilized | | 97.9 | 56.4 | 10.4 | 19.6 | 5.4 |
| % Change | | 0.0 | −4.1 | −2.0 | 8.0 | −3.8 |
| Fresh liquid | S5 Apheresis | 99.0 | 26.5 | 13.0 | 25.7 | 27.0 |
| Lyophilized | | 98.8 | 26.1 | 12.8 | 23.2 | 28.8 |
| % Change | | 0.2 | 1.5 | 1.5 | 9.7 | −6.7 |
| Fresh Liquid | S6 Apheresis | 99.9 | 27.4 | 18.3 | 33.7 | 11.6 |
| Lyophilzed | | 99.8 | 27 | 18.5 | 33.9 | 11.4 |
| % Change | | 0.5 | 0 | 1 | 1 | 0.5 |
| Fresh Liquid | S7 Apheresis | 98.7 | 37.3 | 3.2 | 23.5 | 30.9 |
| Lyophilzed | | 97.8 | 41.8 | 3.1 | 21.8 | 28 |
| % Change | | 0.1 | 0.7 | 8 | 3.4 | 5.3 |
| Fresh Liquid | CytoTrol | 99.8 | 69.7 | 8.9 | 3.9 | 11.1 |
| Lyophilzed | (PCTM) | 99.4 | 68.7 | 9 | 4 | 10.7 |

| | | | | | |
|---|---|---|---|---|---|
| % Change | 0.1 | 0.3 | 1 | 0.6 | 2.9 |

The average frequencies of target populations were compared between the two sources of antibody cocktails. As shown in FIG. 2 and Table 14, the test is reproducible when the antibody reagent changed from fresh liquid format of cocktail to reconstituted lyophilized antibody cocktail, and all the % CVs were within the acceptable range (≤250%).

### Example 3

### 10 Day Stability of Lyophilized Reagents after Resuspending in the Stain Buffer

The lyophilized antibody reagents were determined to be stable for 18 months at room temperature. The product was expected to have 3 months stability after ressuspension of the lyophilized reagents in 400 μL of stain buffer per vial, which could be used for 20 tests/vial. The 10-day stability of the product after resuspension in 2000 μL of stain buffer was also tested.

On Day 1, the lyophilized antibody cocktail was resuspended in 2000 μL of stain buffer. A part was used for Day 1 and the remaining was saved for Day 10. Fresh liquid antibody reagents were separately prepared for comparison. Apheresis samples from four cancer patients and one healthy donor were tested. CytoTrol was used as the positive control testing material (PCTM). CytoTrol consists of a lyophilized human lymphocyte-rich cells with lot-specific reference ranges of surface markers. The results are shown in Table 15.

TABLE 15
DAY−1 stability of lyophilized reagents using cancer patient and healthy donor apheresis
samples.

| | | | | | | | | | % in CD45dim | |
|---|---|---|---|---|---|---|---|---|---|---|
| | | | | % in CD45⁺ | | | | % CD45 | CD19+ | CD19+ |
| Reagent | | | % CD45⁺ in | T | NK | Mono- | B | dim in | CD34+ | CD10+ |
| Type | Sample | Type | Leukocytes | cells | cells | cytes | cells | Leukocytes | Cells | Cells |
| Fresh | 1 | Cancer | 97.1 | 9.6 | 42.3 | 9.3 | 31.0 | 0.2 | NA | NA |
| Liquid | | patient | | | | | | | | |
| lyophilized | | | 97.6 | 11.6 | 41.1 | 9.7 | 29.0 | 0.1 | NA | NA |
| % Change | | | 0.3 | 13.1 | 2.0 | 2.5 | 4.7 | NA | NA | NA |
| Fresh | 2 | Cancer | 99.9 | 0.7 | 0.0 | 3.7 | 93.7 | 0.1 | NA | NA |
| Liquid | | patient | | | | | | | | |
| lyophilized | | | 99.8 | 0.9 | 0.0 | 4.3 | 93.3 | 0.1 | NA | NA |
| % Change | | | 0.0 | NA | NA | 10.5 | 0.3 | NA | NA | NA |
| Fresh | 3 | Cancer | 4.9 | 3.3 | 0.9 | 0.1 | NA | 95.0 | 57.9 | 56.4 |
| Liquid | | patient | | | | | | | | |
| lyophilized | | | 5.2 | 3.8 | 0.9 | 0.0 | NA | 94.6 | 57.3 | 57.2 |
| % Change | | | 4.9 | 9.4 | 0.0 | NA | NA | 0.3 | 0.7 | 1.0 |
| Fresh | 4 | Cancer | 15.8 | 11.2 | 3.4 | 0.0 | 0.7 | 83.8 | 27.9 | 64.2 |
| Liquid | | patient | | | | | | | | |
| lyophilized | | | 15.5 | 11.3 | 3.2 | 0.0 | 0.7 | 84.2 | 29.4 | 68.9 |
| % Change | | | 1.1 | 0.3 | 5.1 | NA | 2.1 | 0.3 | 3.8 | 5.0 |
| Fresh | 5 | Healthy | 99.3 | 41.4 | 7.6 | 24.7 | 18.5 | 0.1 | NA | NA |
| Liquid | | donor | | | | | | | | |
| lyophilized | | | 99.4 | 45.5 | 7.8 | 19.8 | 19.0 | 0.1 | NA | NA |
| % Change | | | 0.1 | 6.6 | 2.1 | 15.6 | 2.1 | NA | NA | NA |
| Fresh | CytoTrol | PCTM | 99.8 | 70.1 | 7.6 | 4.3 | 10.4 | 0.0 | NA | NA |
| Liquid | | | | | | | | | | |
| lyophilized | | | 99.2 | 69.4 | 6.7 | 4.2 | 10.4 | 0.1 | NA | NA |

| | | | | | | | | |
|---|---|---|---|---|---|---|---|---|
| % Change | 0.4 | 0.7 | 9.2 | 2.2 | 0.0 | NA | NA | NA |

TABLE 16
DAY-10 stability of lyophilized reagents using cancer patients and healthy donor
samples

| | | | | | | | | | % in CD45dim | |
|---|---|---|---|---|---|---|---|---|---|---|
| | | | | % in CD45⁺ | | | | % CD45 | CD19+ | CD19+ |
| Reagent | | | % CD45⁺ in | T | NK | Mono- | B | dim in | CD34+ | CD10+ |
| Type | Sample | Study | Leukocytes | cells | cells | cytes | cells | Leukocytes | Cells | Cells |
| Fresh | 1 | Study-1 | 99.4 | 0 | 9.8 | 43.6 | 10.3 | 32.3 | 0 | 0 |
| Liquid | | | | | | | | | | |
| lyophilized | | | 99.4 | 0 | 11.1 | 40.1 | 10. 6 | 32.6 | 0 | 0 |
| % Change | | | 0.0 | NA | 8.8 | 5.9 | 2.0 | 0.7 | NA | NA |
| Fresh | 2 | Study-2 | 99.6 | 0.6 | 0 | 2.4 | 95.5 | 0.2 | 0 | 0 |
| Liquid | | | | | | | | | | |
| lyophilized | | | 99.5 | 0.8 | 0 | 2.4 | 95 | 0.2 | 0 | 0 |
| % Change | | | 0.1 | 20.2 | NA | 0.0 | 0.4 | 0.0 | NA | NA |
| Fresh | 3 | Study-3 | 5.5 | 4.1 | 1 | 0 | 0.1 | 94.3 | 65.6 | 66.4 |
| Liquid | | | | | | | | | | |
| lyophilized | | | 6.3 | 4.6 | 1.3 | 0 | 0.1 | 93.6 | 69.1 | 70.7 |
| % Change | | | 9.6 | 8.1 | 18.4 | NA | NA | 0.5 | 3.7 | 4.4 |
| Fresh | 4 | Study-4 | 15.3 | 84.5 | 0 | 3.4 | 10.8 | 0.6 | 25 | 57.3 |
| Liquid | | | | | | | | | | |
| lyophilized | | | 14.9 | 84.6 | 0 | 3.5 | 10.4 | 0.6 | 24.4 | 62.6 |
| % Change | | | 1.9 | 0.1 | NA | 2.0 | 2.7 | 0.0 | 1.7 | 6.3 |
| Fresh | 5 | Healthy | 99 | 0.1 | 17.8 | 8 | 47.1 | 19.8 | 0 | 0 |
| Liquid | | donor | | | | | | | | |
| lyophilized | | | 99.2 | 0 | 19.4 | 7.4 | 46.7 | 18 | 0 | 0 |
| % Change | | | 0.1 | 141.4 | 6.1 | 5.5 | 0.6 | 6.7 | NA | NA |
| Fresh | CytoTrol | PCTM | 100.0 | 68.9 | 9.5 | 5.2 | 0.0 | 0.0 | 10.2 | 0.0 |
| Liquid | | | | | | | | | | |
| lyophilized | | | 99.9 | 69.2 | 8.6 | 4.2 | 0.0 | 0.0 | 10.6 | 0.0 |

| | | | | | | | | |
|---|---|---|---|---|---|---|---|---|
| % Change | 0.1 | 0.3 | 7.0 | 15.0 | NA | NA | NA | NA |

FIG. 3B and Table 16 indicate the 10-day stability of the lyophilized antibody reagents, with retention of full activity and function compared to fresh liquid antibody reagents with calculated percent change within the acceptable range of ≤25% CV. In addition, the compensation controls were stable for use for 10 days from the day of preparation (data not shown).

### Example 4

### Intermediate Precision-Inter-Analyst Variability

Inter-analyst variability test assesses the ability of an analytical method to operate precisely when executed by multiple analysts. Two apheresis samples and CYTO-TROL were independently tested by two analysts in duplicate. Samples were prepared and analyzed by two analysts independently at the same day using the same lot of CytoTrol (PCTM) and 2 healthy donor samples. The results are shown in Table 17.

TABLE 17
Use of lyophilized reagents, Inter-analyst variability data.

| | | | | % in CD45⁺ | | | |
|---|---|---|---|---|---|---|---|
| Reagent | | | % CD45⁺ in | T | NK | Mono- | B |
| Type | Analyst | Sample | Leukocytes | cells | cells | cytes | cells |
| lyophilized | Analyst1 | S1: Apheresis | 99.8 | 27.0 | 18.5 | 33.9 | 11.4 |
| lyophilized | Analyst2 | | 98.1 | 28.3 | 17.8 | 32.8 | 10.3 |
| % Change | | | 1.3 | 3.3 | 2.7 | 2.4 | 7.6 |
| lyophilized | Analyst1 | S2: Apheresis | 97.8 | 41.8 | 3.1 | 21.8 | 28.0 |
| lyophilized | Analyst2 | | 99.2 | 37.4 | 3.2 | 22.7 | 32.1 |
| % Change | | | 1.0 | 7.9 | 3.6 | 3.0 | 9.7 |
| lyophilized | Analyst1 | CytoTrol | 99.4 | 68.7 | 9.0 | 4.0 | 10.7 |
| lyophilized | Analyst2 | (PCTM) | 99.9 | 68.7 | 9.3 | 3.2 | 10.7 |

| | | | | | |
|---|---|---|---|---|---|
| % Change | 0.4 | 0.0 | 2.6 | 17.2 | 0.0 |

The average frequencies of target populations were compared across the analysts, and % Change was calculated. Table 15 showed that the inter-analyst variability was minimal, and that % CV for all target populations were within the acceptable range (≤25% CV).

### Example 5

### Inter-Assay Precision

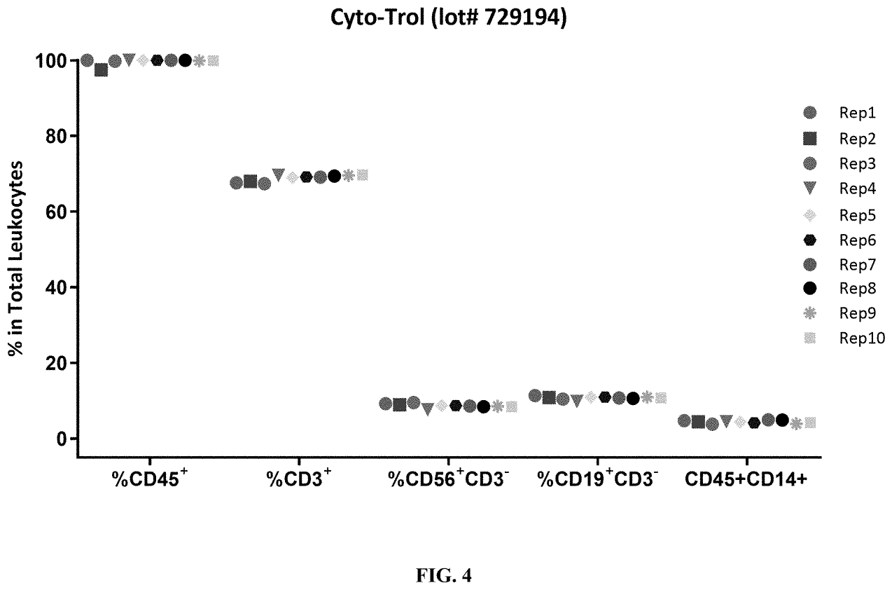

An inter-assay precision test assesses the ability of an analytical method to operate precisely when executed by different analysts in different days. CYTO-TROL (PCTM) was independently tested in 10 different runs. The average frequencies of target populations were compared across the runs, and % Change was calculated. The results are shown in FIG. 4 and Table 18.

TABLE 18
Use of Lyophilized Reagents-INTER Assay Precision

| Cell | | | | | |
|---|---|---|---|---|---|
| Population | % | % | % | % | % |
| (%) | CD45+ | CD3+ | CD56+CD3− | CD19+ | CD14+ |
| Reference | 95-100 | 71-87 | 2.3-10.7 | 5-17 | NA |
| range | | | | | |
| Rep1 | 100 | 67.6 | 9.2 | 11.3 | 4.7 |
| Rep2 | 99.5 | 68 | 8.9 | 10.8 | 4.4 |
| Rep3 | 99.8 | 67.4 | 9.5 | 10.4 | 3.8 |
| Rep4 | 100 | 69.6 | 7.6 | 9.8 | 4.4 |
| Rep5 | 100 | 69 | 8.7 | 10.9 | 4.4 |
| Rep6 | 100 | 69.2 | 8.7 | 10.9 | 4.1 |
| Rep7 | 100 | 69.1 | 8.5 | 10.7 | 4.9 |
| Rep8 | 100 | 69.4 | 8.4 | 10.6 | 4.9 |
| Rep9 | 99.9 | 69.6 | 8.5 | 10.9 | 3.9 |
| Rep10 | 99.9 | 69.7 | 8.4 | 10.7 | 4.2 |
| Average | 99.9 | 68.9 | 8.6 | 10.7 | 4.4 |
| SD | 0.2 | 0.9 | 0.5 | 0.4 | 0.4 |
| % CV | 0.2 | 1.3 | 6 | 3.8 | 8.7 |

FIG. 4 and Tables 18 and 19 showed that the inter-assay precision was optimal, and that % Change for all target populations were within the acceptable range (≤250% CV). All the frequencies of target populations were within the reference ranges of CYTO-TROL lot specific data range (reference range). Typically, the reference ranges in CYTO-TROL are: 95-100% CD45+; 71-87% CD3+; 2.7-11.1% CD56+CD3−; and 5-21% CD19+.

Samples were prepared and analyzed by 2 analysts independently at the same day using the same lot of CytoTrol (PC™) and 2 healthy donor samples.

TABLE 19
USE OF LYOPHILIZED REAGENTS, INTER−ANALYST VARIABILITY

| | | | | % in CD45⁺ | | | |
|---|---|---|---|---|---|---|---|
| Reagent | | | % CD45⁺ in | T | NK | Mono- | B |
| Type | Analyst | Sample | Leukocytes | cells | cells | cytes | cells |
| lyophilized | Analyst1 | S1 | 99.8 | 27.0 | 18.5 | 33.9 | 11.4 |
| lyophilized | Ana1yst2 | | 98.1 | 28.3 | 17.8 | 32.8 | 10.3 |
| % Change | | | 1.3 | 3.3 | 2.7 | 2.4 | 7.6 |
| lyophilized | Analyst1 | S2 | 97.8 | 41.8 | 3.1 | 21.8 | 28.0 |
| lyophilized | Ana1yst2 | | 99.2 | 37.4 | 3.2 | 22.7 | 32.1 |
| % Change | | | 1.0 | 7.9 | 3.6 | 3.0 | 9.7 |
| lyophilized | Analyst1 | CytoTrol | 99.4 | 68.7 | 9.0 | 4.0 | 10.7 |
| lyophilized | Ana1yst2 | (PCTM) | 99.9 | 68.7 | 9.3 | 3.2 | 10.7 |

| | | | | | |
|---|---|---|---|---|---|
| % Change | 0.4 | 0.0 | 2.6 | 17.2 | 0.0 |

### Example 6

### T Test

A two-tailed paired T test was performed comparing 14 data sets from liquid and lyophilized staining performed in different days as shown in Table 20.

TABLE 20
Comparison between liquid cocktails and lyophilized reagents.

| | | | | | | | | | % in CD45 dim | |
|---|---|---|---|---|---|---|---|---|---|---|
| | | | | % in CD45+ | | | | | CD19+ | CD19+ |
| | | Day of | | | NK | Mono- | B- | CD45 | CD34+ | CD10+ |
| # | Study | Reagent | CD45hi | T−cells | Cells | cytes | cells | dim | Cells | Cells |

Liquid Reagents

| | | | | | | | | | | |
|---|---|---|---|---|---|---|---|---|---|---|
| 1 | STUDY-2 | 0 | 99.85 | 0.72 | 0.05 | 3.66 | 93.65 | 0.07 | 0.00 | 0.00 |
| 2 | STUDY-2 | 10 | 99.55 | 0.64 | 0.04 | 2.35 | 95.45 | 0.22 | 0.00 | 0.00 |
| 3 | STUDY-3 | 0 | 86.25 | 34.40 | 20.50 | 0.43 | 26.70 | 10.26 | 5.48 | 6.01 |
| 4 | HEALTHY | 0 | 98.00 | 38.75 | 3.42 | 22.83 | 28.93 | 0.31 | 0.12 | 0.00 |
| | DONOR | | | | | | | | | |
| 5 | STUDY-4 | 10 | 15.50 | 10.98 | 3.44 | 0.03 | 0.64 | 84.10 | 26.43 | 60.73 |
| 6 | STUDY-3 | 10 | 5.19 | 3.69 | 0.94 | 0.03 | 0.11 | 94.63 | 61.70 | 61.40 |
| 7 | HEALTHY | 0 | 99.23 | 41.58 | 7.37 | 24.25 | 18.90 | 0.05 | 0.00 | 0.00 |
| | DONOR | | | | | | | | | |
| | HEALTHY | 10 | 98.98 | 44.93 | 7.49 | 18.15 | 21.70 | 0.14 | 0.00 | 0.00 |
| 8 | DONOR | | | | | | | | | |
| 9 | STUDY-3 | 10 | 99.58 | 85.55 | 2.61 | 0.68 | 0.25 | 0.19 | 0.00 | 0.00 |
| 10 | PCTM | 0 | 99.70 | 69.55 | 8.63 | 3.77 | 11.15 | 0.01 | 0.00 | 0.00 |
| 11 | PCTM | 10 | 99.85 | 69.00 | 9.02 | 4.66 | 10.80 | 0.00 | 0.00 | 0.00 |
| 12 | STUDY-1 | 0 | 97.10 | 9.60 | 42.30 | 9.33 | 30.95 | 0.18 | 0.00 | 0.00 |
| 13 | STUDY-1 | 10 | 99.35 | 10.29 | 43.55 | 8.82 | 32.30 | 0.03 | 0.00 | 0.00 |
| 14 | HEALTHY | 0 | 99.65 | 28.55 | 18.28 | 32.68 | 10.98 | 0.09 | 0.00 | 0.00 |
| | DONOR | | | | | | | | | |

Lyophilized Reagents

| | | | | | | | | | | |
|---|---|---|---|---|---|---|---|---|---|---|
| 15 | STUDY-2 | 0 | 99.80 | 0.93 | 0.04 | 4.25 | 93.30 | 0.09 | 0.00 | 0.00 |
| 16 | STUDY-2 | 10 | 99.45 | 0.79 | 0.04 | 2.43 | 95.00 | 0.17 | 0.00 | 0.00 |
| 17 | STUDY-3 | 0 | 86.40 | 35.45 | 20.95 | 0.32 | 25.10 | 11.30 | 6.89 | 7.33 |
| 19 | HEALTHY | 0 | 98.45 | 39.55 | 3.16 | 22.23 | 30.00 | 0.42 | 0.21 | 0.00 |
| | DONOR | | | | | | | | | |
| 21 | STUDY-4 | 10 | 14.85 | 10.44 | 3.51 | 0.01 | 0.58 | 84.60 | 24.40 | 62.55 |
| 23 | STUDY-3 | 10 | 6.30 | 4.55 | 1.26 | 0.00 | 0.07 | 93.55 | 69.10 | 70.70 |
| 24 | HEALTHY | 0 | 99.63 | 43.23 | 7.31 | 22.80 | 18.53 | 0.05 | 0.00 | 0.00 |
| | DONOR | | | | | | | | | |
| 25 | HEALTHY | 10 | 99.40 | 42.83 | 6.96 | 24.38 | 16.98 | 0.08 | 0.00 | 0.00 |
| | DONOR | | | | | | | | | |
| 26 | STUDY-3 | 10 | 99.73 | 86.18 | 2.56 | 0.70 | 0.21 | 0.08 | 0.00 | 0.00 |
| 27 | PCTM | 0 | 99.60 | 69.05 | 8.33 | 3.98 | 10.65 | 0.05 | 0.00 | 0.00 |
| 28 | PCTM | 10 | 99.85 | 69.30 | 8.56 | 4.02 | 10.90 | 0.01 | 0.00 | 0.00 |
| 29 | STUDY 1 | 0 | 97.55 | 11.55 | 41.10 | 9.66 | 28.95 | 0.09 | 0.01 | 0.01 |
| 30 | STUDY-1 | 10 | 99.35 | 10.55 | 40.05 | 11.10 | 32.55 | 0.04 | 0.00 | 0.00 |
| 31 | HEALTHY | 0 | 98.93 | 27.60 | 18.15 | 33.33 | 10.83 | 0.34 | 0.07 | 0.00 |
| | DONOR | | | | | | | | | |

| | | | | | | | | |
|---|---|---|---|---|---|---|---|---|
| p−value, Paired t−test | | | | | | | | |
| (by Excel) | 0.40 | 0.36 | 0.14 | 0.29 | 0.11 | 0.73 | 0.39 | 0.20 |

Hₒ The mean difference in liquid antibody reagents and lyophilized antibody reagents after staining is 0.
Hₐ The mean difference in liquid antibody reagents and lyophilized antibody reagents after staining is not equal to 0.
Added table 21 summary of the data from T test.

TABLE 21
COMPARABLE METHOD PERFORMANCE TO
LIQUID REAGENTS-PAIRED T-TEST (SUMMARY)

| | Number | | Difference |
|---|---|---|---|
| Reportable attributes | of pairs | P value | (*s or *ns?) |
| CD45hi | 14 | 0.4 | ns |
| T Cells | 14 | 0.36 | ns |
| NK Cells | 14 | 0.14 | ns |
| Monocytes | 14 | 0.29 | ns |
| B Cells | 14 | 0.11 | ns |
| CD45 dim cells | 14 | 0.73 | ns |
| CD45dim/CD19+ CD34+ cells | 14 | 0.39 | ns |
| CD45dim/CD19+ CD10+ cells | 14 | 0.2 | ns |

The average frequencies of target populations were compared across 14 data sets, and % Change was calculated. Table 17 showed that the difference between the means is minimal. The p value of the all target populations were within the acceptable range (p-value >0.05).

The lyophilized cocktail was compared with liquid reagents for isotype, sample, and compensation controls using healthy donors and patient's lots. The comparability of the lyophilized reagents with liquid reagents was confirmed by % difference of frequencies of the parameters, inter-assay precision, and paired T test values. The lyophilized product resuspended in 2000 μL stain buffer showed at least 10-day stability. Single color lyophilized compensation controls were stable for at least 10 days.

### Example 7

### Method Performance Parameters

The method described above met all expected performance parameters, based on, for example, Sconochia G. Et al. Leukemia 2005; 19:69-76 and Van Acker HH et al. Frontiers in Immunology 2017; 8:892. These performance parameters may be summarized as follows:

TABLE 21
Exemplay Method Performance Parameters

| Method Parameter | | Recommended Criteria |
|---|---|---|
| Accuracy (Recovery) | | 80% to 120% |
| Linearity | | R2 > 0.96; % CV ≤ 25 |
| LLOQ | | % CV ≤ 25%; Accuracy |
| | | 20% to 120% |
| Precision | Repeatability | % CV ≤ 25 |
| | (Intra-assay) | (30% at LLOQ) |
| | Intermediate: | % CV ≤ 25 |
| | Interanalyst | (30% at LLOQ) |
| | Intermediate: | % CV ≤ 25 |
| | Inter-assay | (30% at LLOQ) |
| Robustness | Antibody cocktail | % CV ≤ 25 |
| | volume | 30% at LLOQ); |
| | TVC seeded/well | % Difference ≤ 20 |
| | CD45+ acquisition | |
| | events | |
| | Antibody staining | |
| | time | |

### Example 8

### Method Specificity

The method sensitivity was studied thoroughly to ensure optimal analytical performance. Specificity measures the extent to which a test is specific for the target populations of interest and is measured by comparing known % target population in a sample to the % population detected by the test. The specificity for CD34, CD19, and CD56 conjugated antibodies were examined by testing known positive and negative samples for the corresponding markers. For CD34 antibody specificity, Stem-Trol (commercially sourced/manufactured CD34+ positive control cells) from StemCell Technologies was used as the positive sample. Similarly, MAVER-1/MRL3008 (CD19+ B cell line), and pure NK cells (from StemCell Technologies) were used as positive samples for the specificity of CD19 and CD56 antibodies, respectively. CD34+ cells, CD19+ cells and NK cells percentages are the output measurements for this assessment. The method PCTM, CYTO-TROL, was also used in the specificity test, as it has lot-specific reference ranges provided by the manufacturer.

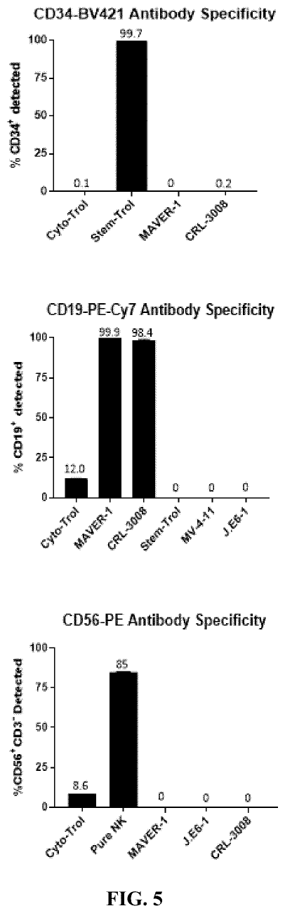

FIG. 5 and Table 21 show that the method successfully detected the test materials at their expected values: negative result for negative test material, positive result for positive test material, and within the reference ranges for CYTO-TROL. Overall, the method is specific for the detection of CD34⁺ cells, CD19⁺ cells and NK cells.

TABLE 21
Samples used in the specificity study and their expected and detected values for
% CD34⁺, % CD19⁺ and % CD56⁺CD3⁻ NK cells

| | | % CD34⁺ | | % CD19⁺ | | % CD56⁺CD3⁻ NK | |
|---|---|---|---|---|---|---|---|
| Test Material | Sample Type | Expected | Detected | Expected | Detected | Expected | Detected |
| CYTO-TROL | PCTM | 0 | 0.0 | 11 ± 6 | 12 | 6.5 ± 4.2 | 8.7 |
| MAVER-1 | CD19⁺ B cell | 0 | 0.0 | 100 | 99.9 | 0 | 0.0 |
| | line | | | | | | |
| Stem-Trol | Manufactured | 100 | 99.8 | 0 | 0.0 | none | none |
| | CD34⁺ cells | | | | | | |
| MV-4-11 | Macrophage | none | none | 0 | 0.0 | none | none |
| | cell line | | | | | | |
| Jurkat.E6-1 | T cell line | none | none | 0 | 0.0 | 0 | 0.0 |
| Pure NK | purified NK | none | none | none | none | 85 | 85.0 |
| cells | cells | | | | | | |

### Example 9

### Method Accuracy

Accuracy of a method describes the closeness of the test results obtained by the method to an accepted reference value (obtained by previously accepted methods). The accuracy or recovery of the CD3⁻ impurity method in frequency was assessed with the PCTM, CYTO-TROL, which has reference ranges provided by its manufacturer. Three independent experiments were conducted on different days. The average frequency from three experiments were calculated. % Accuracy was determined as:

$$\% Accuracy = 100 * \frac{Tested Average}{Reference Average}$$

Table 21 shows the testing results of CYTO-TROL lot #729188 compared to the lot-specific reference ranges for frequencies of CD45⁺, CD3⁺, CD56⁺ CD3⁻, and CD19⁺ provided by the manufacturer. The three experiments showed good precision (% CV≤20) and accuracy (% Accuracy within 80%-120%), and all listed populations are within the reference ranges.

TABLE 23
Testing results of CYTO-TROL control cells

| Cell | | | | |
|---|---|---|---|---|
| Population (%) | % CD45⁺ | % CD3⁺ | % CD56⁺CD3⁻ | % CD19⁺ |
| Reference range¹ | 98 ± 3 | 79 ± 8 | 6.9 ± 4.2 | 13 ± 8 |
| Experiment 1 | 98 | 74.4 | 7.9 | 10.5 |
| Experiment 2 | 97.5 | 74.8 | 8 | 10.7 |
| Experiment 3 | 98 | 74.1 | 8.5 | 10.7 |
| Average (Exp 1-3) | 97.8 | 74.4 | 8.1 | 10.6 |
| % CV (Exp 1-3) | 0.3 | 0.5 | 4.0 | 1.1 |
| % Accuracy | 99.8 | 94.2 | 117.9 | 81.8 |

### Example 10

### Method Robustness

The method described in the above examples is also robust. Robustness assesses the reliability of an analytical method and is a measure of the method's capacity to produce similar data under small but deliberate variations in method parameters. Method robustness for this method may be evaluated, for example, in the following aspects:

Antibody staining/incubation time: Incubation duration times are tested for antibody staining from 10 minutes to 45 minutes. Any incubation time ≥45 minutes is not necessary for surface staining and is considered not efficient in the testing workflow.

Total TVC seeded/well and lower limit of acquisition event: Seeding cell number per well for staining at 0.5×10⁶ or 1×10⁶ TVC.

To determine the size of the sample that will provide a given precision when detecting small or rare populations a method as described in Maecker HT et al. Cytometry Part A 2006 (69A): 1037-1042; ICSH/ICCS Workgroup. Cytometry B Clin Cytometry. 2013; 84:279-357; or Allan A L et al. Journal of Oncology 2010 (2010:426218 may be used.

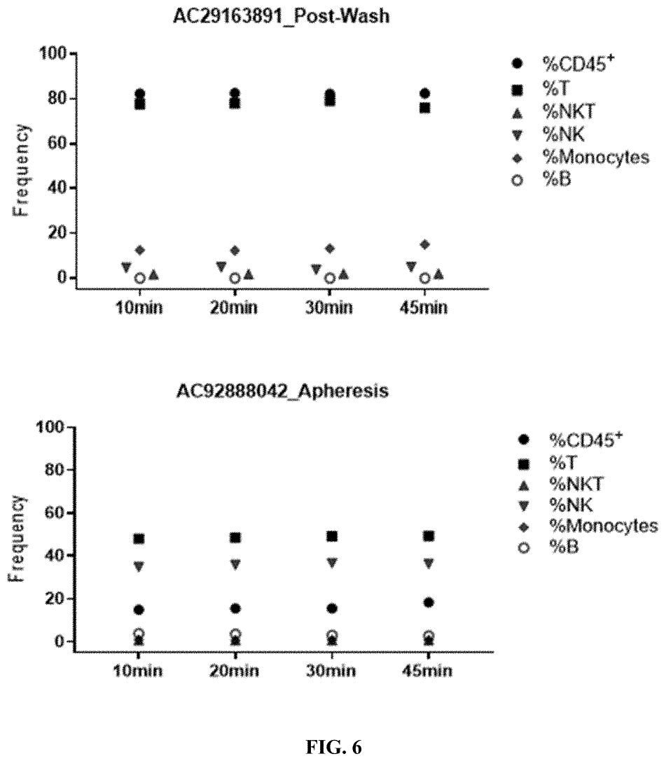

The robustness of this method was tested for antibody staining incubation time at 10, 20, 30, and 45 minutes. Two samples were run for each time point and the detected frequencies of following target cell populations are compared: % CD45⁺ in leukocytes, % subsets (CD3⁺ T cells, CD56⁺ CD3⁺ NKT cells, CD56⁺ CD3⁻ NK cells, CD14⁺ CD3⁻ Monocytes and CD19⁺ CD3⁻ B cells) in CD45⁺ cells. FIG. 6 and Table 21 show that all target cell populations are comparable among the ranges of incubation time tested. % CV was less than 25% for all target cell populations. Therefore, a dynamic incubation time range of 10 minutes to 45 minutes was established. 100 μL of staining volume was used for all samples.

TABLE 21
Method robustness: antibody staining incubation time

| | Antibody | | | | | | | | | |
|---|---|---|---|---|---|---|---|---|---|---|
| | Incubation | | | | CD3⁺ | | CD3⁻ | | | |
| | Time | | | CD3⁺ | CD56⁺ | CD3⁻ | CD56⁺ | CD14⁺ | CD19⁺ | |
| Sample | (minute) | % Leukocyte | CD45⁺ | of % CD45⁺ | | | | | | CD45_{dim} |
| STUDY 2 | 10 | 15.3 | 15.2 | 63.9 | 2.1 | 36.1 | 6.7 | 25.1 | 0.0 | 0.0 |
| SAMPLE 1 | 20 | 12.0 | 11.9 | 65.7 | 2.2 | 34.3 | 7.4 | 24.6 | 0.0 | 0.0 |
| POST WASH | 30 | 13.7 | 13.6 | 64.8 | 2.1 | 35.2 | 7.0 | 24.9 | 0.0 | 0.0 |
| | 45 | 15.6 | 15.0 | 61.4 | 2.8 | 38.7 | 8.2 | 28.7 | 0.0 | 0.0 |
| | mean | 14.1 | 13.9 | 63.9 | 2.3 | 36.1 | 7.3 | 25.8 | 0.0 | 0.0 |
| | stdev | 1.7 | 1.5 | 1.9 | 0.3 | 1.9 | 0.7 | 1.9 | 0.0 | 0.0 |
| | % CV | 11.8% | 11.0% | 2.9% | 14.2% | 5.2% | 8.9% | 7.4% | N/A | N/A |
| STUDY 2 | 10 | 45.9 | 7.3 | 48.8 | 1.0 | 51.2 | 34.0 | 1.5 | 4.1 | 38.2 |
| SAMPLE 2 | 20 | 44.8 | 7.8 | 47.9 | 0.9 | 52.1 | 34.7 | 2.2 | 3.6 | 36.6 |
| Apheresis | 30 | 47.2 | 8.6 | 48.0 | 1.2 | 52.1 | 34.2 | 3.3 | 3.7 | 38.5 |
| | 45 | 46.3 | 8.6 | 48.7 | 1.2 | 51.3 | 33.6 | 4.2 | 3.3 | 37.6 |
| | mean | 46.0 | 8.1 | 48.3 | 1.1 | 51.7 | 34.1 | 2.8 | 3.7 | 37.7 |
| | stdev | 1.0 | 0.7 | 0.5 | 0.1 | 0.5 | 0.5 | 1.2 | 0.4 | 0.8 |
| | % CV | 2.1% | 8.2% | 1.0% | 12.6% | 0.9% | 1.3% | 42.2% | 9.6% | 2.2% |
| CYTO−TROL | 20 | 99.8 | 98.1 | 75.2 | 5.08 | 24.7 | 7.52 | 3.01 | 11.9 | NA |
| positive control | 30 | 99.8 | 98.2 | 75.6 | 5.57 | 24.3 | 7.26 | 3.34 | 11.6 | NA |
| cells | 45 | 99.7 | 97.7 | 75.5 | 5.49 | 24.5 | 7.45 | 3.09 | 12.2 | NA |
| | mean | 99.8 | 98.0 | 75.4 | 5.4 | 24.5 | 7.4 | 3.1 | 11.9 | NA |
| | stdev | 0.1 | 0.3 | 0.2 | 0.3 | 0.2 | 0.1 | 0.2 | 0.3 | NA |
| | % CV | 0.1% | 0.3% | 0.3% | 4.9% | 0.8% | 1.8% | 5.5% | 2.5% | NA |

The method sets limits for TVC seeded per well that will provide a given precision when detecting small or rare subpopulations such as non-T cells in final product or T cells in highly tumor-burden patient starting materials. Based on relevant guidance for clinical flow cytometry methods a simple calculation was implemented to determine the size of the sample that will provide a given precision when detecting small or rare subpopulations as seen below:

$$r = {( \frac{100}{CV} )}^{2} ,$$

where r is the number of events that meet the required criteria while CV is the desired coefficient of variation.

Based on this calculation, a desirable % CV at 500 provides a good level of confidence and precision. Therefore,

$$r = {( \frac{100}{CV} )}^{2} = {( \frac{100}{5} )}^{2} = 400.$$

And, the lowest limit for such detectability quality among the LLOQ values of the established critical reporting parameters is 0.2%. By extrapolation, a minimum of 0.04×10⁶ TVC need to be acquired to obtain 400 such residual cells. Using this approach, a minimum of 50,000 TVC should be acquired if an acceptable % CV is 15%, because (100/15)²/0.2%=22,222. A basic study was performed using 2 starting patient materials and stained with the full antibody panel for CD45⁺ acquisition events at 10,000, 20,000, 50,000, 100,000, and 200,000 events. Various acquisition events were compared against each other by % CV as shown in Table 22 and Table 23. Data listed in Table 22 were found not to have significant impacts when comparing among 50,000, 100,000, and 200,000 acquisition events. This is a potential variable with smaller value than 1% when collect 50,000 events. The results listed in Table 23, where lower % B cells data (just equal to or greater than LLOQ value) are impacted, with 10,000 and 20,000 collected CD45⁺ events, yet this has no impact on B-blasts parameters.

On the same day, a simple TVC seeding experiment was performed with 1×10⁶ and 1×10⁵ TVC using 3 different samples for a rough seeding limit estimate assessment. Data shown in Table 24 provided comparability of all reporting parameters in frequencies between both TVC seeding/well tests. Also shown in Table 24 are the findings that % B cells is below LLOQ in sample apheresis lot AC25809161.

Taken together, when this method is set to the acquisition stop criteria when viable CD45+ cells reaches 150,000 events is sufficient to achieve a minimum of calculated number of 22,222 events. The stopping gate at 100,000 viable CD45+ events guarantees the minimum cell events needed to detect lower frequency cell populations at LLOQ of 0.2% with a CV≤15%. Further, this will not make the cell acquisition too time-consuming and inefficient for the testing workflow. To account for the potential cell loss during cell staining and washing steps, the original cell seeding density and TVC per well is set at 1E6 per well.

TABLE 22
Method robustness: Various viable CD45⁺ acquisition events collected data

| | | Acquisition | | | NKT | Non−T | NK | CD14⁺ | B |
|---|---|---|---|---|---|---|---|---|---|
| | | Events | | T Cells | Cells | Cells | Cells | Monocyte | Cells |
| Lot No | Sample Type | (k = 1000) | CD45⁺ | of % CD45⁺ | | | | | |
| C19-19-046 | Apheresis | 50 K | 99.3 | 54.2 | 9.59 | 45.1 | 9.06 | 28.32 | 0.01 |
| | | 100 K | 99.3 | 54.4 | 9.68 | 44.8 | 8.78 | 28.57 | 0.02 |
| | | % Difference | 0 | 0.4% | 0.9% | −0.7% | −3.2% | 0.9% | 45.8% |
| | | 50 K | 99.3 | 54.2 | 9.59 | 45.1 | 9.06 | 28.32 | 0.01 |
| | | 200 K | 99.2 | 57.3 | 10.1 | 41.9 | 9.07 | 25.61 | 0.02 |
| | | % Difference | 0 | 5.4% | 5.0% | −7.6% | 0.1% | 40.6% | 31.6% |
| | | 100 K | 99.3 | 54.4 | 9.68 | 44.8 | 8.78 | 28.57 | 0.02 |
| | | 200 K | 99.2 | 57.3 | 10.1 | 41.9 | 9.07 | 25.61 | 0.02 |
| | | % Difference | 0 | 5.1% | 4.2% | −6.9% | 3.2% | −11.6% | −26.3% |
| C19-19-047 | Apheresis | 50 K | 99.7 | 69.9 | 9.0 | 29.7 | 1.2 | 19.0 | 0.04 |
| | | 100 K | 99.7 | 71.7 | 8.8 | 28.0 | 1.2 | 17.5 | 0.05 |
| | | % Difference | 0 | 2.5% | −2.3% | −6.1% | −1.6% | −8.6% | 2.2% |
| | | 50 K | 99.7 | 69.9 | 9.0 | 29.7 | 1.2 | 19.0 | 0.04 |
| | | 200 K | 99.7 | 72.1 | 9.0 | 27.6 | 1.2 | 17.3 | 0.04 |
| | | % Difference | 0 | 3.1% | −0.3% | −7.6% | −4.2% | −10.2% | 0.0% |
| | | 100 K | 99.7 | 71.7 | 8.8 | 28.0 | 1.2 | 17.5 | 0.05 |
| | | 200 K | 99.7 | 72.1 | 9.0 | 27.6 | 1.2 | 17.3 | 0.04 |
| | | % Difference | 0 | 0.6% | 1.9% | 4.4% | −2.5% | −1.6% | −2.3% |
| C1919046 | Post−thawed | 100 K | 99.4 | 58.8 | 7.53 | 40.6 | 1.54 | 27.24 | 0.06 |
| | | 200 K | 99.4 | 59.4 | 7.56 | 40 | 1.5 | 26.67 | 0.04 |
| | | % Difference | 0 | 1.0% | 0.4% | 4.5% | −2.7% | −2.1% | −54.1% |
| C19−19−048 | Apheresis | 100 K | 99.3 | 69.1 | 6.24 | 30.2 | 5.44 | 10.68 | 10.7 |
| | | 200 K | 99.3 | 69.5 | 6.1 | 29.9 | 5.5 | 10.17 | 10.5 |
| | | % Difference | 0 | 0.6% | −2.3% | 4.0% | 1.1% | −5.0% | −1.9% |

Note:
lower values statistically have higher % CV when they are compared against each other; the values of % B cells are all <LLOQ (<0.2%)

TABLE 23
Method robustness: Lower viable CD45⁺ acquisition events stopping gate data and 10 K CD45⁺ is not comparable for % CD14⁺ monocyte
measurement

| | CD45+ | | | | | | | | | | | |
|---|---|---|---|---|---|---|---|---|---|---|---|---|
| | Events | | | | | | | CD14⁺ | | | | |
| | Acquired | Total | | | | Non-T | NK | Mono- | | | CD19⁺CD10⁺ | CD19⁺CD34⁺ |
| Sample | (1 K = 1,000) | Leukocyte | CD45⁺ | T cells | NKT | cells | cells | cyte | B cells | CD45^{dim} | (CD45^{dim}) | (CD45^{dim}) |
| AC8326348 | 50 K | 99.5 | 98.7 | 62.6 | 8.0 | 29.5 | 1.6 | 24.2 | 0.1 | N/A | N/A | N/A |
| frozen ALL | 20 K | 99.2 | 96.2 | 63.5 | 8.2 | 28.3 | 1.4 | 22.5 | 0.3 | N/A | N/A | N/A |
| Apheresis | Mean | 99.4 | 97.4 | 63.1 | 8.1 | 28.9 | 1.5 | 23.3 | 0.2 | | | |
| | SD | 0.2 | 1.8 | 0.6 | 0.2 | 0.8 | 0.1 | 1.2 | 0.1 | | | |
| | % CV | 0.2% | 1.8% | 1.0% | 2.0% | 2.8% | 7.5% | 5.0% | 49.3% | | | |
| | 50 K | 99.5 | 98.7 | 62.6 | 8.0 | 29.5 | 1.6 | 24.2 | 0.1 | N/A | N/A | N/A |
| | 10 K | 99.4 | 97.1 | 67.5 | 8.6 | 23.9 | 1.5 | 18.3 | 0.2 | N/A | N/A | N/A |
| | mean | 99.5 | 97.9 | 65.1 | 8.3 | 26.7 | 1.5 | 21.2 | 0.2 | | | |
| | SD | 0.1 | 1.1 | 3.5 | 0.4 | 3.9 | 0.1 | 4.2 | 0.0 | | | |
| | % CV | 0.1% | 1.2% | 5.3% | 5.4% | 14.7% | 4.1% | 19.7% | 13.7% | | | |
| ALL03001 | 50 K | 99.9 | 20.6 | 58.0 | 6.4 | 35.7 | 17.6 | 1.9 | 11.4 | 78.2 | 94.6 | 94.2 |
| fresh ALL | 20 K | 99.6 | 20.1 | 58.2 | 6.1 | 35.8 | 17.8 | 1.6 | 11.5 | 78.9 | 93.0 | 92.4 |
| PBMC | Mean | 99.7 | 20.4 | 58.1 | 6.2 | 35.7 | 17.7 | 1.8 | 11.4 | 78.5 | 93.8 | 93.3 |
| | SD | 0.2 | 0.4 | 0.1 | 0.2 | 0.1 | 0.1 | 0.2 | 0.0 | 0.5 | 1.2 | 1.3 |
| | % CV | 0.2% | 1.7% | 0.2% | 3.5% | 0.3% | 0.6% | 9.9% | 0.3% | 0.6% | 1.2% | 1.4% |
| | 50 K | 99.9 | 20.6 | 58.0 | 6.4 | 35.7 | 17.6 | 1.9 | 11.4 | 78.2 | 94.6 | 94.2 |
| | 10 K | 99.8 | 22.4 | 52.1 | 5.2 | 42.8 | 16.8 | 0.7 | 10.2 | 76.9 | 93.4 | 92.6 |
| | Mean | 99.9 | 21.5 | 55.0 | 5.8 | 39.2 | 17.2 | 1.3 | 10.8 | 77.6 | 94.0 | 93.4 |
| | SD | 0.1 | 1.3 | 4.2 | 0.8 | 5.0 | 0.6 | 0.8 | 0.8 | 0.9 | 0.9 | 1.1 |
| | % CV | 0.1% | 5.9% | 7.6% | 14.4% | 12.8% | 3.3% | 61.4% | 7.7% | 1.2% | 0.9% | 1.2% |

Note:
lower values statistically have higher % CV when they are compared against each other; LLOQ of % B cells is 0.2%

TABLE 24
Method robustness: TVC/well for plating

| | | | | | | | | | | | CD19⁺ | CD19⁺ |
|---|---|---|---|---|---|---|---|---|---|---|---|---|
| | | Leuko- | CD45⁺ | T | NKT | Non-T | NK | CD14⁺ | B | | CD10⁺ | CD34⁺ |
| | | cytes (of | (Of % Leu- | Cells | Cells | Cells | Cells | Monocyte | Cells | CD45^{dim} | (CD45^{dim}) | (CD45^{dim}) |
| Sample | Cell# | live cell) | kocytes) | Of % Leukocytes | | | | | | | Of % CD45^{dim} | |
| AC25809161 | 1E+06 | 100 | 97.6 | 61.4 | 8.3 | 30.3 | 1.5 | 25.0 | 0.0 | N/A | N/A | N/A |
| fresh DLBCL | 1E+05 | 99.5 | 98.7 | 62.6 | 8.0 | 29.5 | 1.6 | 24.2 | 0.1 | N/A | N/A | N/A |
| Apheresis | Mean | 99.7 | 98.1 | 62.0 | 8.2 | 29.9 | 1.5 | 24.6 | 0.1 | | | |
| | Stdev | 0.3 | 0.7 | 0.9 | 0.2 | 0.6 | 0.0 | 0.6 | 0.1 | | | |
| | % CV | 0.3% | 0.8% | 1.4% | 2.9% | 2.0% | 3.0% | 2.3% | 141% | | | |
| | % Difference | 0.5% | −1.1% | −2.0% | 4.0% | 2.8% | −4.3% | 3.2% | N/A | | | |
| ALL03001 | 1E+06 | 99.9 | 20.6 | 58.0 | 6.4 | 35.7 | 17.6 | 1.9 | 11.4 | 78.2 | 94.6 | 94.2 |
| fresh B−ALL | 1E+05 | 99.8 | 20.5 | 57.6 | 6.0 | 36.5 | 17.4 | 1.8 | 12.0 | 78.4 | 94.4 | 94.0 |
| PBMC | Mean | 99.8 | 20.6 | 57.8 | 6.2 | 36.1 | 17.5 | 1.8 | 11.7 | 78.2 | 94.6 | 94.2 |
| | Stdev | 0.1 | 0.1 | 0.3 | 0.3 | 0.6 | 0.1 | 0.1 | 0.4 | 0.1 | 0.1 | 0.1 |
| | % CV | 0.1% | 0.3% | 0.5% | 4.8% | 1.7% | 0.8% | 4.7% | 3.6% | 0.1% | 0.1% | 0.2% |
| | % Difference | 0.2% | 0.5% | 0.7% | 6.6% | −2.4% | 1.1% | 6.4% | −5.3% | −0.2% | 0.2% | 0.2% |
| CYTO-TROL | 1E+06 | 99.2 | 99.1 | 70.0 | 4.5 | 25.4 | 7.5 | 2.7 | 12.9 | N/A | N/A | N/A, |
| | 1E+05 | 98.3 | 97.6 | 69.4 | 4.0 | 26.3 | 7.1 | 3.4 | 12.8 | N/A | N/A | N/A |
| | Mean | 98.8 | 98.4 | 69.7 | 4.3 | 25.9 | 7.3 | 3.0 | 12.8 | | | |
| | Stdev | 0.6 | 1.1 | 0.5 | 0.3 | 0.6 | 0.3 | 0.4 | 0.1 | | | |
| | % CV | 0.6% | 1.1% | 0.7% | 8.0% | 2.5% | 4.1% | 14.4% | 0.6% | | | |
| | % Difference | 0.9% | 1.5% | 0.9% | 10.8% | −3.5% | 5.6% | −22.7% | 0.8% | | | |

Note:
Statistically, lower values have higher % CV when they are compared against each other; LLOQ of % B cells is 0.2%

### Example 11

### Stability of SINGLE COLOR Lyophilized Compensation Controls

Compensation controls stability was one day after reconstitution with stain buffer (BSA) according to the manufacturer. At Kite we evaluated the stability of compensation controls for 14 days. Seven lyophilized compensation controls, CD3-APC, CD10-FITC, CD14-PerCPCy5.5, CD19-PECy7, CD34-BV421, CD45-V500 and CD56-PE are included in the lyophilized compensation control kit (BD catalog #625812). Stain buffer (BSA) 300 μL and a drop of UltroComp eBeads (compensation beads) are added to each lyophilized compensation control tube and incubate for 15 mintues to prepare compensation controls for the assay. Lyophilized compensation control tubes were saved for 14 days at 4° C. after the reconstitution and MFIs were calculated to assess stability.

TABLE 25
FOURTEEN DAYS STABILITY OF SINGLE
COLOR LYOPHILIZED COMPENSATION CONTROLS

| Compensation | Day 1 (MFI) | Day 14 (MFI) | |
|---|---|---|---|
| Control | (18 Sep. 2020) | (2 OCT. 2020) | % Change |
| CD3-APC | 25574 | 25498 | −0.3 |
| CD10-FITC | 8461 | 9035 | 6.3 |
| CD14-PerCPCy5.5 | 5635 | 6745 | −16.5 |
| CD19-PECy7 | 4832 | 6205 | −22.1 |
| CD34-BV421 | 7515 | 7346 | 2.3 |
| CD45-V500 | 4812 | 4904 | −1.9 |
| CD56-PE | 3498 | 3895 | −10. |

Table 25 shows the MFI of the reconstituted lyophilized single color compensation controls from Day 0 and Day 14 are comparable. Data from day 14 lyopilized compensation controls show <25% percent difference with day 0 fresh single color compensation controls. This shows the stability of compensation control stored at 4° C. for 14 days.

What is claimed is:

1. A method of simultaneous identifying two or more of lymphocytes, NK-T cells, NK cells, monocytes, early B cell progenitor cell, or combinations thereof in a cell population, comprising simultaneously detecting the presence or absence of two or more of lymphocytes, NK-T cells, NK cells, monocytes, and/or early B cell progenitor cells using two or more of the markers on the surface of these cells as described in Table 2, optionally with one or more of the fluorescently-labeled antibodies as described in Tables 3, 4, and 5, using fluorescence detection methods.

2. A method of assessing the non-CD3+ contaminants in a population of cells comprising primarily CD4+ and/or CD8+ T cells comprising contacting the population of cells with one or more antibodies against specific surface markers for lymphocytes, NK-T cells, NK cells, monocytes, and/or early B cell progenitor cell to create a mixture, wherein two or more of the specific cell surface markers are described in Table 2, optionally, wherein the one or more antibodies are selected from Tables 3, 4, and 5, and analyzing the mixture for the distribution of cells with specific cell surface markers by fluorescence detection methods.

3. A method of treating cancer in a subject by immunotherapy in need thereof, comprising administering to the subject a T cell preparation wherein one or more of the CD3-impurities (e.g., NK-T cells, NK cells, monocytes, early B cell progenitor cell, or combinations thereof) in the T cell preparation is characterized by the method of claim 2.

4. The method of claim 3, wherein the T cell preparation is autologous, optionally from a cancer patient or a healthy donor.

5. The method of claim 3, wherein the T cell preparation is allogeneic, optionally from a cancer patient or a heathy donor.

6. The method of claim 3, wherein the T cells are engineered with a CAR or T cell receptor.

7. A method for determining whether a T cell product is suitable for immunotherapy, comprising characterizing one or more of the CD3− cell impurities (e.g., NK-T cells, NK cells, monocytes, early B cell progenitor cell, or combinations thereof) in the T cell product using one of the antibodies or cocktail of antibodies described in Tables 3, 4, and 5, and determining whether the T cell product is suitable based on the levels of CD3− cell impurities in the T cell product.

8. The method of claim 7, wherein the acceptable levels are set by regulatory authorities (e.g., FDA, EMEA, etc).

9. An assay or a kit for identifying at least one of T lymphocytes, NK-T cells, NK cells, monocytes total lymphocytes, early B cell progenitor cell, or combinations thereof in a blood cell population using one or more of the antibodies or cocktails of antibodies described in Tables 3, 4, and 5.

10. The assay or kit of claim 9, wherein the assay or kit is used to characterize the presence of CD3− cells in T cell products for immunotherapy.

11. The kit of claim 10, wherein the kit comprises (a) one or more antibodies to detect one or more cell surface markers for any one or more of these cells (see, e.g., Table 2) and (2) reagents to carry on the binding of the antibody with the cell surface markers, and, optionally, (3) instructions for using the reagents for the kit's purpose.

12. The method of claim 11, wherein the antibodies (two or more) are all lyophilized together in the same container.

13. A composition comprising a panel of fluorescently-labeled antibodies for identifying the presence or absence of T cells, NK-T cells, NK cells, monocytes, early B cell progenitor cell, or combinations thereof cells in a cell population, comprising two or more antibodies against two or more of the cell surface markers identified in Table 2, optionally wherein one or more of the antibodies are described in Tables 3, 4, or 5.

14. A composition comprising immune cells fluorescently stained with the composition of claim 13.

15. The composition of claim 13, wherein the composition comprises all of the antibodies in Tables 3, 4, or 5, optionally, together with a cell viability marker.

16. The composition of claim 13, wherein the composition comprises antibodies against all of the cell surface markers in Table 2, optionally, together with a cell viability marker.

17. The composition of claim 13, wherein the composition comprises all of the antibodies described in Table 6 in the same amounts of Table 6 or in identical multiples of such amounts.
